SEQUENCE LISTING (80 sequences)

SEQ ID NO: 1 - DNA, 21 bp, Artificial sequence
  Synthetic primer
ccagcatttctgcctttgtga

SEQ ID NO: 2 - DNA, 20 bp, Artificial sequence
  Synthetic primer
ccagcatttctgcctgtttg

SEQ ID NO: 3 - DNA, 17 bp, Artificial sequence
  Synthetic primer
cagctcctcccagattt

SEQ ID NO: 4 - DNA, 18 bp, Artificial sequence
  Synthetic primer
acctgccggtactccagg

SEQ ID NO: 5 - DNA, 20 bp, Artificial sequence
  Synthetic primer
acctgccggtactccaggta

SEQ ID NO: 6 - DNA, 20 bp, Artificial sequence
  Synthetic primer
gcccttggaccccacaggaa

SEQ ID NO: 7 - DNA, 24 bp, Artificial sequence
  Synthetic primer
aggactttcacatagctggtttca

SEQ ID NO: 8 - DNA, 22 bp, Artificial sequence
  Synthetic primer
ggactttcacatagctggtttc

SEQ ID NO: 9 - DNA, 19 bp, Artificial sequence
  Synthetic primer
tttattcagatttaatttc

SEQ ID NO: 10 - DNA, 20 bp, Artificial sequence
  Synthetic primer
caagagacatgatgactctc

SEQ ID NO: 11 - DNA, 20 bp, Artificial sequence
  Synthetic primer
ttcctcaggcttgcagtgca

SEQ ID NO: 12 - DNA, 19 bp, Artificial sequence
  Synthetic primer
gagaggaggaggaggtggc

SEQ ID NO: 13 - DNA, 20 bp, Artificial sequence
  Synthetic primer
gatctgttgacccagcagtg

SEQ ID NO: 14 - DNA, 18 bp, Artificial sequence
  Synthetic primer
cactgggttgcctctgtc

SEQ ID NO: 15 - DNA, 19 bp, Artificial sequence
  Synthetic primer
ctgggttgcctctgtcgag

SEQ ID NO: 16 - DNA, 19 bp, Artificial sequence
  Synthetic primer
gggttgcctctgtcgagtg

SEQ ID NO: 17 - DNA, 18 bp, Artificial sequence
  Synthetic primer
ggctgctggaaccctcac

SEQ ID NO: 18 - DNA, 20 bp, Artificial sequence
  Synthetic primer
gcttggcccctcctcttcac

SEQ ID NO: 19 - DNA, 20 bp, Artificial sequence
  Synthetic primer
gaacaaggactccaggatac

SEQ ID NO: 20 - DNA, 21 bp, Artificial sequence
  Synthetic primer
catacatgcattcctcagggt

SEQ ID NO: 21 - DNA, 21 bp, Artificial sequence
  Synthetic primer
gaactttctctgcagctgggc

SEQ ID NO: 22 - DNA, 20 bp, Artificial sequence
  Synthetic primer
tggcagggtttctagggtct

SEQ ID NO: 23 - DNA, 20 bp, Artificial sequence
  Synthetic primer
ggtttccccgaatactcctg

SEQ ID NO: 24 - DNA, 20 bp, Artificial sequence
  Synthetic primer
ttgctaggatgatggcactg

SEQ ID NO: 25 - DNA, 20 bp, Artificial sequence
  Synthetic primer
ccaagatgtcctggtccttg

SEQ ID NO: 26 - DNA, 18 bp, Artificial sequence
  Synthetic primer
cagcacacccaccagatc

SEQ ID NO: 27 - DNA, 18 bp, Artificial sequence
  Synthetic primer
agagtctcgggatcgtgc

SEQ ID NO: 28 - DNA, 18 bp, Artificial sequence
  Synthetic primer
agtctcgggatcgtgcag

SEQ ID NO: 29 - DNA, 18 bp, Artificial sequence
  Synthetic primer
tctcgggatcgtgcagca

SEQ ID NO: 30 - DNA, 19 bp, Artificial sequence
  Synthetic primer
atgcagcgaagcagagtct

SEQ ID NO: 31 - DNA, 18 bp, Artificial sequence
  Synthetic primer
cctttcagcgatgcagcg

SEQ ID NO: 32 - DNA, 18 bp, Artificial sequence
  Synthetic primer
gtatgcacggctactggc

SEQ ID NO: 33 - DNA, 20 bp, Artificial sequence
  Synthetic primer
gctatctgagccgtctagac

SEQ ID NO: 34 - DNA, 20 bp, Artificial sequence
  Synthetic primer
aatgttacgagcagtgatgc

SEQ ID NO: 35 - DNA, 18 bp, Artificial sequence
  Synthetic primer
tggggccctcgtggaatg

SEQ ID NO: 36 - DNA, 21 bp, Artificial sequence
  Synthetic primer
cagttaatgggcatcgttaag

SEQ ID NO: 37 - DNA, 20 bp, Artificial sequence
  Synthetic primer
atctgtgccccacaaaccag

SEQ ID NO: 38 - DNA, 18 bp, Artificial sequence
  Synthetic primer
ggcccgggatgtaggcac

SEQ ID NO: 39 - DNA, 20 bp, Artificial sequence
  Synthetic primer
ggtaatcactccccagatag

SEQ ID NO: 40 - DNA, 18 bp, Artificial sequence
  Synthetic primer
ctcccggggtaatcactc

SEQ ID NO: 41 - DNA, 18 bp, Artificial sequence
  Synthetic primer
cagtctcccggggtaatc

SEQ ID NO: 42 - DNA, 18 bp, Artificial sequence
  Synthetic primer
tgaggaggcaaggcagtc

SEQ ID NO: 43 - DNA, 21 bp, Artificial sequence
  Synthetic primer
ggattggttacattcaaaggc

SEQ ID NO: 44 - DNA, 21 bp, Artificial sequence
  Synthetic primer
agagtgattcgcgtgggtacc

SEQ ID NO: 45 - DNA, 22 bp, Artificial sequence
  Synthetic primer
ggctgcaacggcggaagaaaac

SEQ ID NO: 46 - DNA, 19 bp, Artificial sequence
  Synthetic primer
tgcaacggcggaagaaaac

SEQ ID NO: 47 - DNA, 20 bp, Artificial sequence
  Synthetic primer
atgtctggtaacggcaatgc

SEQ ID NO: 48 - DNA, 23 bp, Artificial sequence
  Synthetic primer
ttgcagatggctccgatggtgaa

SEQ ID NO: 49 - DNA, 18 bp, Artificial sequence
  Synthetic primer
ggctccgatggtgaagcc

SEQ ID NO: 50 - DNA, 22 bp, Artificial sequence
  Synthetic primer
ttgggtgaaagacaacagcatc

SEQ ID NO: 51 - DNA, 21 bp, Artificial sequence
  Synthetic primer
gtagagttcggccgaaggaac

SEQ ID NO: 52 - DNA, 21 bp, Artificial sequence
  Synthetic primer
caggagctgggcaatgaagac

SEQ ID NO: 53 - DNA, 22 bp, Artificial sequence
  Synthetic primer
cattgaaggagaagatctgcct

SEQ ID NO: 54 - DNA, 22 bp, Artificial sequence
  Synthetic primer
gagtagaagaggaagaagcggt

SEQ ID NO: 55 - DNA, 18 bp, Artificial sequence
  Synthetic primer
gaagccggcccaggctcg

SEQ ID NO: 56 - DNA, 20 bp, Artificial sequence
  Synthetic primer
gatgactctggtcagggcaa

SEQ ID NO: 57 - DNA, 21 bp, Artificial sequence
  Synthetic primer
caccaaggagaagatctgcct

SEQ ID NO: 58 - DNA, 21 bp, Artificial sequence
  Synthetic primer
tcctcagtagtaggagcctgt

SEQ ID NO: 59 - DNA, 20 bp, Artificial sequence
  Synthetic primer
ctacagacacagtgggtcgc

SEQ ID NO: 60 - DNA, 22 bp, Artificial sequence
  Synthetic primer
gcttggtattagaggatagcag

SEQ ID NO: 61 - DNA, 20 bp, Artificial sequence
  Synthetic primer
tccgtgaggaggcaaggttc

SEQ ID NO: 62 - DNA, 22 bp, Artificial sequence
  Synthetic primer
atcggattgactccagagagta

SEQ ID NO: 63 - DNA, 20 bp, Artificial sequence
  Synthetic primer
tagagttcggccgaaggaac

SEQ ID NO: 64 - DNA, 20 bp, Artificial sequence
  Synthetic primer
ctgggcaatgaagacccaca

SEQ ID NO: 65 - DNA, 22 bp, Artificial sequence
  Synthetic primer
cattgaaggagaagatctgcct

SEQ ID NO: 66 - DNA, 20 bp, Artificial sequence
  Synthetic primer
caggcttgcagtgctgactc

SEQ ID NO: 67 - DNA, 19 bp, Artificial sequence
  Synthetic primer
ggctcggtgaggaggcaag

SEQ ID NO: 68 - DNA, 20 bp, Artificial sequence
  Synthetic primer
gatgactctggtcagggcaa

SEQ ID NO: 69 - DNA, 21 bp, Artificial sequence
  Synthetic primer
caccaaggagaagatctgcct

SEQ ID NO: 70 - DNA, 21 bp, Artificial sequence
  Synthetic primer
caggcttgcagtgctgactct

SEQ ID NO: 71 - DNA, 21 bp, Artificial sequence
  Synthetic primer
atctgacaagagtccaggttc

SEQ ID NO: 72 - DNA, 19 bp, Artificial sequence
  Synthetic primer
cgctgacgctttggagctc

SEQ ID NO: 73 - DNA, 20 bp, Artificial sequence
  Synthetic primer
tccgtgaggaggcaaggttc

SEQ ID NO: 74 - DNA, 19 bp, Artificial sequence
  Synthetic primer
gagcctgcgcacccaccaa

SEQ ID NO: 75 - DNA, 18 bp, Artificial sequence
  Synthetic primer
acctgccggtactccagg

SEQ ID NO: 76 - DNA, 18 bp, Artificial sequence
  Synthetic primer
tggggccctcgtggaatg

SEQ ID NO: 77 - DNA, 21 bp, Artificial sequence
  Synthetic primer
agagtgattcgcgtgggtacc

SEQ ID NO: 78 - DNA, 22 bp, Artificial sequence
  Synthetic primer
ttgggtgaaagacaacagcatc

SEQ ID NO: 79 - DNA, 27 bp, Artificial sequence
  Synthetic primer
agtggacctggttgttcactccttgaa

SEQ ID NO: 80 - DNA, 23 bp, Artificial sequence
  Synthetic primer
acctgcccactgtgcttcctcct

(19) United States
(12) Patent Application Publication
(10) Pub. No.: US 2016/0208338 A1
(43) Pub. Date: Jul. 21, 2016

(54) Method of Treating a Patient or Exempting a Patient From Further Treatment Subsequent to Tumor Removal

(71) Applicant: Peter Kufer, Moosburg (DE)

(72) Inventor: Peter Kufer, Moosburg (DE)

(21) Appl. No.: 15/001,348

(22) Filed: Jan. 20, 2016

Related U.S. Application Data
(60) Provisional application No. 62/106,034, filed on Jan. 21, 2015.

(51) Int. Cl. C12Q 1/68

(52) U.S. Cl. CPC C12Q 1/6886; C12Q 2600/158; C12Q 2600/16; C12Q 2600/118

(57) ABSTRACT

Described is method of treating a patient or exempting a patient from further treatment subsequent to tumor removal such as surgery. The method involves performing a highly sensitive real-time PCR for specific detection of transcripts for more than one MAGE gene and a reference gene such as porphobilinogen desaminase (PBGD), glyceraldehyd-3-phosphate dehydrogenase (GAPDH), beta-2-microglobin or beta-actin in a tissue sample of a tumor patient.

This application claims the benefit of U.S. Provisional Patent Application No. 62/106,034, filed Jan. 21, 2015, the entirety of which is incorporated herein by reference.

### INCORPORATION OF SEQUENCE LISTING

The sequence listing that is contained in the file named “DEBEP0133US_ST25.txt”, which is 16 KB (as measured in Microsoft Windows®) and was created on Jan. 13, 2016, is filed herewith by electronic submission and is incorporated by reference herein.

### BACKGROUND OF THE INVENTION

Among frequent solid tumors with more than 100,000 new cases per year in the US are lung cancer, colorectal cancer, prostate cancer and breast cancer. Lung cancer, for example, is the leading cause of cancer-related death in Europe and the United States¹. Non-small cell lung cancer (NSCLC) accounts for approximately 80% of all cases². The 5-year survival of patients with resected stage IA NSCLC is only 73% and drops to 24% in resected stage IIIA NSCLC³.

In solid tumors, the frequent cause of cancer-related death after tumor removal by, e.g., surgery, chemotherapy or radiation is the development of distant metastases originating from the outgrowth of minimal residual disease (MRD) caused by dissemination of cancer cells prior to tumor removal, that remain undetected by conventional staging procedures. However, quantitative measurement of MRD predicting disease free-survival and/or clinical recurrence in individual patients reliably enough to base therapeutic decisions on has been established only in a few malignancies such as B-lineage acute lymphoblastic leukemia (B-ALL). More than 90% of adult patients with B-ALL in complete hematological remission, who failed to clear MRD from bone marrow as determined by real-time quantitative polymerase chain reaction (PCR) develop a hematological relapse⁴. Availability of PCR markers highly specific for the malignant cells such as individual rearrangements of immunoglobulin genes was a key success factor for the advancement of MRD assessment from an explorative method into an established staging procedure for clinical patient management.

For MRD assessment in solid tumors, particularly in NSCLC, colorectal cancer, prostate cancer and breast cancer several members of family A of melanoma-associated antigens (MAGE-A) are available as PCR markers of similarly high tumor specificity^{5, 25}. The MAGE-A gene family has 12 members (MAGE-A1 to -A12) located on chromosome Xq28^{6,7} MAGE-A family members are normally restricted in their adult tissue expression to testis and placenta⁸ and expressed briefly during early embryonic development⁹. In tumor cells, genome-wide epigenetic reprogramming frequently leads to activation of MAGE-A expression through promoter hypo-methylation¹¹. In addition, other chromatin remodeling events like histone acetylation and methylation further modulate MAGE-A expression. While little is understood of the physiological function of MAGE-A proteins, there is more clarity on their role in promoting malignancy. MAGE-A proteins interfere with two major tumor suppressor mechanisms: By suppressing p53-mediated transcription they inhibit both p53-mediated apoptosis and senescence¹¹. Moreover, by targeting the p53 pathway, MAGE-A proteins confer resistance to chemotherapeutic drugs that act via p53-mediated apoptosis¹².

Overall, single members of the MAGE-A family instead of a combination of several or all members of the MAGE-A family are expressed too sporadically to be suitable as the only marker for MRD assessment. However, quantitative measurement of minimal residual disease (MRD) predicting clinical recurrence in individual cancer patients reliably enough to base therapeutic decisions on is available only in very few indications such as B-lineage acute lymphoblastic leukemia (B-ALL) but still missing in solid tumors including resected non-small cell lung cancer (NSCLC), colorectal cancer, prostate cancer and breast cancer.

### SUMMARY OF THE INVENTION

The problem of the present invention was thus to provide a method for measuring the risk of a metastatic relapse. The problem is solved by the subject-matter as set forth in the appended claims.

### BRIEF DESCRIPTION OF THE DRAWINGS

In the following a brief description of the appended figures will be given. The figures are intended to illustrate the present invention in more detail. However, they are not intended to limit the subject matter of the invention to any extent.

FIG. 1. FIG. 1 shows schematically the course of Kaplan-Meier estimates of distant-relapse-free survival in percentage among patients with a MAGE-A expression level ≧0.01 versus <0.01 in blood and bone marrow samples against the follow-up after surgery (in days).

FIGS. 2A-2D. FIG. 2A shows schematically the course of Kaplan-Meier estimates of distant-relapse-free survival in percentage among patients with a MAGE-A expression level ≧0.2 versus <0.2 in blood and bone marrow samples against the follow-up after surgery (in days). FIG. 2B shows schematically the course of Kaplan-Meier estimates of overall survival in percentage among patients with a MAGE-A expression level ≧0.2 versus <0.2 in blood and bone marrow samples against the follow-up after surgery (in days). FIG. 2C shows schematically the course of Kaplan-Meier estimates of cancer-free survival in percentage among patients with a MAGE-A expression level ≧0.2 versus <0.2 in blood and bone marrow samples against the follow-up after surgery (in days). FIG. 2D shows schematically the course of Kaplan-Meier estimates of local-relapse-free survival in percentage among patients with a MAGE-A expression level ≧0.2 versus <0.2 in blood and bone marrow samples against the follow-up after surgery (in days).

FIGS. 3A-3C. FIG. 3A shows schematically the course of Kaplan-Meier estimates of overall survival in percentage among patients with a MAGE-A expression level ≧0.2 versus <0.2 in solely bone marrow samples against the follow-up after surgery (in days). FIG. 3B shows schematically the course of Kaplan-Meier estimates of cancer-free survival in percentage among patients with a MAGE-A expression level ≧0.2 versus <0.2 in solely bone marrow samples against the follow-up after surgery (in days). FIG. 3C shows schematically the course of Kaplan-Meier estimates of distant-relapse-free survival in percentage among patients with a MAGE-A expression level ≧0.2 versus <0.2 in solely bone marrow samples against the follow-up after surgery (in days).

### DETAILED DESCRIPTION

Because of its quantitative nature, the highly sensitive real-time RT-PCR of the invention is particularly useful for measuring the load of disseminated tumor cells in individual patients, thus estimating the risk of a metastatic relapse originating from the early tumor cell spread that took place prior to successful treatment of the primary tumor. Thus, the method of the invention helps to decide more precisely on the requirement of an adjuvant tumor therapy than is possible with diagnostic methods of the prior art.

Accordingly, the present invention refers to a method of treating a patient or exempting a patient from further treatment subsequent to tumor removal such as surgery comprising the following steps:
a) performing a highly sensitive real-time PCR for specific detection of transcripts (mRNA) of more than one MAGE gene and a real-time PCR for specific detection of transcripts (mRNA) of a reference gene such as porphobilinogen desaminase (PBGD), glyceraldehyd-3-phosphate dehydrogenase (GAPDH), beta-2-microglobin or beta-actin in a tissue sample of a tumor patient, wherein reverse transcription of mRNA into cDNA of more than one MAGE gene and of the reference gene prior to real-time PCR is carried out in the same cDNA synthesis reaction
b) performing said highly sensitive real-time PCR for more than one MAGE gene and said real-time PCR for a reference gene such as porphobilinogen desaminase (PBGD), glyceraldehyd-3-phosphate dehydrogenase (GAPDH), beta-2-microglobin or beta-actin in at least one calibrator sample
c) quantifying the expression level of said MAGE genes in said tissue sample of a tumor patient by calculating the calibrator normalized relative ratio, according to the following formula:

$$Normalized Ratio = \frac{\frac{{N}_{To ( S )}}{{N}_{Ro ( S )}}}{\frac{{N}_{To ( C )}}{{N}_{Ro ( C )}}} = \frac{\frac{{E}_{T}^{CpT ( C )}}{{E}_{R}^{CpR ( C )}}}{\frac{{E}_{T}^{CpT ( S )}}{{E}_{R}^{CpR ( S )}}} = \frac{{E}_{T}^{CpT ( C )}}{{E}_{R}^{CpR ( C )}} \times \frac{{E}_{R}^{CpR ( S )}}{{E}_{T}^{CpT ( S )}} = {E}_{T}^{CpT ( C ) - CpT ( S )} \times {E}_{R}^{CpR ( S ) - CpR ( C )}$$

N_{To}/N_{Ro}: Initial number of target/reference molecules, i.e., of MAGE cDNAs/reference cDNA
CpT: Cycle number at target detection threshold (crossing point)
CpR: Cycle number at reference detection threshold (crossing point)
E_{T}: Efficiency of target amplification
E_{R}: Efficiency of reference amplification
T: Target (i.e., MAGE gene(s))
R: Reference (i.e., reference gene)
S: Tissue sample of a tumor patient (i.e., patient sample)
C: Calibrator sample
d) performing a subsequent therapy, if the resulting MAGE gene expression level of step c) is equal to or above a threshold value selected from the interval of 0.01 to 1.0, preferably selected from the interval of 0.05 to 0.5, more preferably selected from the interval of 0.1 to 0.3 and most preferably is equal to or above a threshold value of 0.2, and/or
e) exempting patients from a subsequent therapy, if the resulting MAGE gene expression level of step c) is below a threshold value selected from the interval of 0.01 to 1.0, preferably selected from the interval of 0.05 to 0.5, more preferably selected from the interval of 0.1 to 0.3 and most preferably is below 0.2.

In a preferred embodiment the at least one calibrator sample used in the method according to the present invention (i) is any tissue of a healthy subject spiked with a defined number of tumor cells, preferably blood, bone marrow, lymph nodes, body fluids, e.g., urine, stool or sputum, or other secondary organs, and/or (ii) is subjected to reverse transcription of mRNA into cDNA of more than one MAGE gene and of the reference gene in the same cDNA synthesis reaction followed by said highly sensitive real-time PCR for more than one MAGE gene and said real-time PCR for a reference gene.

In another preferred embodiment, the at least one calibrator sample used in the method according to the present invention is spiked with a defined number of tumor cells of the human melanoma cell line Mz2-Mel or the human sarcoma cell line LB23-SAR.

In another preferred embodiment, the at least one calibrator sample used herein for determining the MAGE-A1, -A2, -A3/6, -A10 and -A12 gene expression level consisted of two mL of whole blood from a healthy human donor spiked with 10 tumor cells of the human melanoma cell line Mz2-Mel (Ludwig Institute of Cancer Research, Brussels, Belgium [26]) cultured according to the instructions of the cell line provider.

In another preferred embodiment, the calibrator sample used herein for determining the MAGE-A4 gene expression level consisted of two mL of whole blood from a healthy human donor spiked with 10 tumor cells of the human sarcoma cell line LB23-SAR (Ludwig Institute of Cancer Research, Brussels, Belgium [27]) cultured according to the instructions of the cell line provider.

According to the present invention the highly sensitive real-time RT-PCR is performed in that in a first step total RNA is isolated from the tissue of the tumor patient and from the calibrator sample, e.g., by phenol/chloroform extraction.

The tissue of the tumor patient, i.e., patient sample may be any tissue of the tumor patient, preferably blood, bone marrow, lymph nodes, body fluids, e.g., urine, stool or sputum, or other secondary organs.

Spiking of defined numbers of tumor cells from cultured cell lines into whole blood from healthy donors can be carried out without undue burden by the skilled person, e.g., by using micromanipulation devices known in the art and/or commercially available.

The total RNA of the tissue of the tumor patient and the total RNA of the calibrator sample are then used as templates in two separate cDNA-synthesis reactions, i.e., reverse transcription reactions. In a first reverse transcription reaction the total RNA of the tissue of the tumor patient is transcribed by the enzyme reverse transcriptase into cDNA and in a second reverse transcription reaction the total RNA of the calibrator sample is transcribed by the enzyme reverse transcriptase into cDNA. In each of these two separate reverse transcription reactions MAGE cDNAs of more than one MAGE gene and reference cDNA of a reference gene such as porphobilinogen desaminase (PBGD), glyceraldehyd-3-phosphate dehydrogenase (GAPDH), beta-2-microglobin or beta-actin are produced simultaneously, i.e., in the same reaction tube by using specific primers. These primers enable the generation of MAGE cDNAs and reference cDNA, respectively by annealing to specific sequences of the respective MAGE-mRNAs and reference mRNA, respectively in the total RNA.

The amount of MAGE cDNAs represents the expression level of the MAGE genes. The MAGE gene expression levels are determined by using the Light Cycler quantification analysis software (e.g., LightCycler Relative Quantification Software, Roche Molecular Biochemicals, Mannheim, Germany) by which a normalized ratio may be determined using the following formula:

$$Normalized Ratio = \frac{\frac{{N}_{To ( S )}}{{N}_{Ro ( S )}}}{\frac{{N}_{To ( C )}}{{N}_{Ro ( C )}}} = \frac{\frac{{E}_{T}^{CpT ( C )}}{{E}_{R}^{CpR ( C )}}}{\frac{{E}_{T}^{CpT ( S )}}{{E}_{R}^{CpR ( S )}}} = \frac{{E}_{T}^{CpT ( C )}}{{E}_{R}^{CpR ( C )}} \times \frac{{E}_{R}^{CpR ( S )}}{{E}_{T}^{CpT ( S )}} = {E}_{T}^{CpT ( C ) - CpT ( S )} \times {E}_{R}^{CpR ( S ) - CpR ( C )}$$

N_{To}/N_{Ro}: Initial number of target/reference molecules, i.e., of MAGE cDNAs/reference cDNA
CpT: Cycle number at target detection threshold (crossing point)
CpR: Cycle number at reference detection threshold (crossing point)
E_{T}: Efficiency of target amplification
E_{R}: Efficiency of reference amplification
T: Target (i.e., MAGE gene(s))
R: Reference (i.e., reference genes)
S: Tissue sample of a tumor patient (i.e., patient sample)
C: Calibrator sample

This highly sensitive real-time RT-PCR is described in detail in EP 1 565 577 B1. Briefly, a real-time MAGE PCR, i.e., PCR-amplification of reverse transcribed MAGE-cDNA, in accordance with the present invention, can be implemented either by using a sequence unspecific DNA-dye like SYBR Green I or by applying sequence-defined fluorescent probes for the detection of specific amplicons. For carrying out the latter method, the sequences of MAGE mRNA molecules have to be screened for unique marker-defining regions if the detection of individual MAGE parameters is desired or pan-MAGE specific areas if the detection of several MAGE markers is desired in a single reaction. This unique hybridization region on the sequence must be located in between two oligonucleotides used as primers for the PCR and should neither be self-complementary, monotonous, or repetitive nor complementary to the PCR primers. The application of TaqMan probes requires the design of a single double-labeled fluorescent probe, for the application of hybridization probes the design of two fluorescent oligonucleotides is needed that hybridize in close proximity (1 to 5 bases) to each other on the amplicon to enable the distance-dependent transfer of energy between the fluorophores (fluorescence resonance energy transfer (FRET)). The reaction conditions have to be carefully evaluated and optimized, involving the adaptation of primer and probe concentrations, temperatures and duration of PCR-cycling, etc.

In a preferred embodiment of the method of the invention the MAGE genes serving as markers in a highly sensitive real-time RT-PCR are selected from the functional genes of MAGE subfamilies A, B and/or C (Table 1).

TABLE 1
Members of the MAGE gene family showing restricted expression
in malignant tumors and testicular germ cells only.

| Gene Subfamily | Gene Name |
|---|---|
| MAGE-A | hMAGE-A1 |
| | hMAGE-A2 |
| | hMAGE-A3 |
| | hMAGE-A4 |
| | hMAGE-A5 |
| | hMAGE-A6 |
| | hMAGE-A8 |
| | hMAGE-A9 |
| | hMAGE-A10 |
| | hMAGE-A11 |
| | hMAGE-A12 |
| MAGE-B | hMAGE-B1 |
| | hMAGE-B2 |
| | hMAGE-B3 |
| | hMAGE-B4 |
| | hMAGE-B5 |
| | hMAGE-B6 |
| | hMAGE-B10 |
| | hMAGE-B16 |
| | hMAGE-B17 |
| MAGE-C | hMAGE-C1 |
| | hMAGE-C2 |
| | hMAGE-C3 |
| | hMAGE-C4 |

Except for the pseudogenes, expression of the members of these MAGE-subfamilies is highly restricted to tumor cells, while they are completely silent in normal adult tissue with the only exception of testicular germ cells. Thus expression of functional MAGE A, B and/or C genes as detected by the highly sensitive real-time RT-PCR is highly indicative of the systemic spread of cancer cells from the primary tumor. The RT-PCR of the method of the present invention may be performed in any tissue of the tumor patient, preferably in blood, bone marrow, lymph nodes or other secondary organs.

Besides blood and bone marrow many kinds of body fluids or tissues, like urine, stool or sputum, are easily accessible to search for malignant cells. The real-time MAGE RT-PCR of the present invention is therefore also applicable as highly sensitive screening tool in secondary tumor prevention and can achieve the early detection of neoplasia particularly in individuals who are highly at risk of developing cancer.

In a particularly preferred embodiment of the method of the present invention the MAGE genes serving as markers comprise MAGE-A 1, 2, 3, 4, 6, 10 and/or 12. These genes are most frequently expressed in many different types of tumors of various histological origins.

In another preferred embodiment of the method of the invention at least one primer for reverse transcription of MAGE mRNA is selected from the following groups of oligonucleotides:

| | | SEQ |
|---|---|---|
| primer | sequence (5′-3′) | ID NO: |
| (A) | | |
| MgRT1a | CCA GCA TTT CTG CCT TTG TGA | 1 |
| MgRT1b | CCA GCA TTT CTG CCT GTT TG | 2 |
| MgRT2 | CAG CTC CTC CCA GAT TT | 3 |
| MgRT3a | ACC TGC CGG TAC TCC AGG | 4 |
| MgRT3b | ACC TGC CGG TAC TCC AGG TA | 5 |
| MgRT4 | GCC CTT GGA CCC CAC AGG AA | 6 |
| MgRT5a | AGG ACT TTC ACA TAG CTG GTT TCA | 7 |
| MgRT5b | GGA CTT TCA CAT AGC TGG TTT C | 8 |
| MgRT6 | TTT ATT CAG ATT TAA TTT C | 9 |
| (B) | | |
| Mg1_RT1 | CAA GAG ACA TGA TGA CTC TC | 10 |
| Mg1_RT2 | TTC CTC AGG CTT GCA GTG CA | 11 |
| Mg1_RT3 | GAG AGG AGG AGG AGG TGG C | 12 |
| Mg1_RT4 | GAT CTG TTG ACC CAG CAG TG | 13 |
| Mg1_RT5a | CAC TGG GTT GCC TCT GTC | 14 |
| Mg1_RT5c | CTG GGT TGC CTC TGT CGA G | 15 |
| Mg1_RT5d | GGG TTG CCT CTG TCG AGT G | 16 |
| Mg1_RT5e | GGC TGC TGG AAC CCT CAC | 17 |
| Mg1_RT6 | GCT TGG CCC CTC CTC TTC AC | 18 |
| Mg1_RT7 | GAA CAA GGA CTC CAG GAT AC | 19 |

Primers for reverse transcription as depicted in group A, are perfectly matching with each of the mRNA-sequences of MAGE-A 1, 2, 3, 4, 6, 10 and 12. However, despite the perfect match, some of these RT-primers alone may not lead to detection of expression of certain MAGE-A family members, e.g., MAGE-A 1 with sufficient sensitivity by real-time RT-PCR under standard conditions as provided, e.g., by the manufacturer of the LightCycler System. Under these recommended conditions weaknesses in detection of certain MAGE-A family members may be compensated by combining two primers of group A with each other or in case of weakness in detection of MAGE-A1 by combining a group A-primer with one of the group B-primers, which are monospecific for the cDNA-synthesis of MAGE-A 1 only. Depending on which at least two different members of the MAGE-A group encoding target antigens for cytotoxic T cells (i.e., MAGE-A 1, 2, 3, 4, 6, 10 and 12) are to be detected by real-time RT-PCR, different single RT-primers or combinations of RT-primers of group A and/or B may be applicable. In any individual case, however, in accordance with the present invention, careful testing of candidate RT-primers is required to end up with an optimal choice allowing the expression of the selected MAGE genes to be detected by real-time RT-PCR with a high level of sensitivity. As pointed out above, testing of RT-primers for the highly sensitive real-time MAGE RT-PCR has to be carried out in the presence of the whole cocktail of RT-primers during cDNA-synthesis, in order to cope with the unpredictable interferences among different RT-primers.

In addition to the reverse transcription of MAGE transcripts, reverse transcription of a reference mRNA is carried out in a cDNA-synthesis reaction followed by real-time RT-PCR amplification of reference cDNAs. In order to be capable of serving as reference the corresponding marker (in the following referred to as “reference marker”) most preferably is an essentially non-inducible gene. It is further preferred that the expression level of the reference gene is constant in essentially all cells. Furthermore, in accordance with the present invention, it is critical that a specific cDNA-primer for reverse transcription (RT) of the reference mRNA is used as integral member of the RT-primer cocktail comprising the MAGE-specific cDNA-primers to guarantee equal assay conditions for both the different MAGE transcripts to be analyzed and the reference marker.

In a preferred embodiment of the method of the present invention the reference gene is porphobilinogen desaminase (PBGD), glyceraldehyd-3-phospat dehydrogenase (GAPDH), beta-2-microglobin or beta-actin.

In a further preferred embodiment of the method of the present invention the primer for reverse transcription of PBGD mRNA is selected from the following group of oligonucleotides:

| | | SEQ |
|---|---|---|
| primer | Sequence (5′-3′) | ID NO: |
| PBGD_RT2 | CAT ACA TGC ATT CCT CAG GGT | 20 |
| PBGD_RT3 | GAA CTT TCT CTG CAG CTG GGC | 21 |
| PBGD_RT4 | TGG CAG GGT TTC TAG GGT CT | 22 |
| PBGD_RT10a | GGT TTC CCC GAA TAC TCC TG | 23 |
| PBGD_RT10d | TTG CTA GGA TGA TGG CAC TG | 24 |
| PBGD_RT12b | CCA AGA TGT CCT GGT CCT TG | 25 |
| PBGD_RT12c | CAG CAC ACC CAC CAG ATC | 26 |
| PBGD_RT12d | AGA GTC TCG GGA TCG TGC | 27 |
| PBGD_RT12e | AGT CTC GGG ATC GTG CAG | 28 |
| PBGD_RT12f | TCT CGG GAT CGT GCA GCA | 29 |
| PBGD_RT12g | ATG CAG CGA AGC AGA GTC T | 30 |
| PBGD_RT12h | CCT TTC AGC GAT GCA GCG | 31 |
| PBGD_RT13a | GTA TGC ACG GCT ACT GGC | 32 |
| PBGD_RT14a | GCT ATC TGA GCC GTC TAG AC | 33 |
| PBGD_RT15a | AAT GTT ACG AGC AGT GAT GC | 34 |
| PBGD_RT15b | TGG GGC CCT CGT GGA ATG | 35 |
| PBGD_RT15e | CAG TTA ATG GGC ATC GTT AAG | 36 |
| PBGD_RT15f | ATC TGT GCC CCA CAA ACC AG | 37 |
| PBGD_RT15g | GGC CCG GGA TGT AGG CAC | 38 |
| PBGD_RT15h | GGT AAT CAC TCC CCA GAT AG | 39 |
| PBGD_RT15i | CTC CCG GGG TAA TCA CTC | 40 |
| PBGD_RT15j | CAG TCT CCC GGG GTA ATC | 41 |
| PBGD_RT15k | TGA GGA GGC AAG GCA GTC | 42 |
| PBGD_RT15l | GGA TTG GTT ACA TTC AAA GGC | 43 |

In another embodiment of the method of the present invention the PCR-primers for amplification of PBGD-cDNA comprise oligonucleotides selected from the following groups:

| PBGD sense primer | sequence (5′-3′) | SEQ ID NO: |
|---|---|---|
| hu_PBGD_se | AGA GTG ATT CGC GTG GGT ACC | 44 |
| PBGD_8 | GGC TGC AAC GGC GGA AGA AAA C | 45 |
| PBGD_8_F | TGC AAC GGC GGA AGA AAA C | 46 |
| PBGD_ATG-Eco | ATG TCT GGT AAC GGC AAT GC | 47 |
| PBGD antisense | | |
| primer | sequence (5′-3′) | SEQ ID NO: |
| PBGD_3 | TTG CAG ATG GCT CCG ATG GTG AA | 48 |
| PBGD_3.1_R | GGC TCC GAT GGT GAA GCC | 49 |
| PBGD_R | TTG GGT GAA AGA CAA CAG CAT C | 50 |

In an even more preferred embodiment of the method of the present invention oligonucleotides hu_PBGD_se and PBGD_3.1_R or hu_PBGD_se and PBGD_R are used as primer pairs for PCR-amplification of PBGD-cDNA.

In another preferred embodiment of the method of the present invention the oligonucleotides MgRT3a and Mg1_RT5a are used as primers for reverse transcription of MAGE-A mRNA in the cDNA-synthesis reaction.

In a most preferred embodiment of the method of the present invention oligonucleotides MgRT3a and PBGD_RT15b are used as primers for reverse transcription in the cDNA-synthesis reaction.

In another embodiment of the method of the present invention the MAGE- and/or the reference marker-PCR are nested or semi-nested PCRs. In order to achieve the desired high sensitivity for detection of mRNA transcribed by rare tumor cells from more than one MAGE gene, the real-time RT-PCR may be designed as nested or semi-nested PCR. For this purpose a first round of cDNA-amplification may be carried out with an appropriate pair of PCR-primers either by conventional or real-time PCR. Most preferably, this first round of PCR should not proceed to the plateau phase of amplification. Otherwise, quantification of the template content in the sample to be analyzed by the method of the present invention may become very difficult or even impossible. Moreover, it may be preferable to stop such a first round of PCR in the early or middle linear phase of amplification instead of proceeding to the late linear phase, in order to avoid interferences of an excess of preamplified PCR-products with the subsequent round of real-time PCR. Accordingly, the number of PCR-cycles and the reaction conditions that are appropriate for such a preamplification step have to be carefully optimized, respectively. In particular these parameters should be adapted to the distribution of template amounts in the collection of samples to be analyzed, to make sure, on the one hand, that the level of high sensitivity of the method of the invention is sufficient to detect MAGE in those samples showing very weak expression and, on the other hand, that quantification of MAGE in other samples showing higher expression is still feasible.

In a particularly preferred embodiment of the method of the present invention PCR-primers are used comprising pairs of oligonucleotides specifically amplifying only a single member of the selected group of MAGE genes, respectively. Despite the high homology among different members of the MAGE gene family, making the design of such monospecific oligonucleotides more difficult, a highly sensitive real-time MAGE RT-PCR for detecting the individual expression of more than one MAGE gene is highly preferable. Only thus, a quantitative expression profile of individual MAGE genes of rare disseminated tumor cells in individual cancer patients can be obtained, which may be essential for the selection of those members of the MAGE family to be included, e.g., in an optimal tumor vaccine for treating patients subsequent to tumor removal (e.g., after tumor surgery).

In another embodiment of the method of the present invention PCR-primers are used comprising pairs of oligonucleotides amplifying more than one member of the selected group of MAGE genes, respectively (=pan-MAGE PCR).

Following reverse transcription real-time PCR amplification of MAGE cDNA with such consensus primers, like those suggested by Park et al. (2002) may be carried out, which make use of the high level of sequence homology among the different MAGE gene transcripts. For detection of the real-time PCR-amplification product(s) the sequence-independent SYBR green I method can be applied using the LightCycler System; alternatively, sequence-specific fluorescent probes, e.g., TaqMan or hybridization probes may be used. Furthermore, tissue samples (e.g., bronchoscopic biopsies) from cancer patients with different types of tumors (e.g., non-small cell lung (NSCL) cancer) may be analyzed accordingly. For this embodiment of the method of the invention it is of particular advantage that the particular way of cDNA-synthesis disclosed by the present invention makes sure that each single member of the MAGE family selected as a marker for the highly sensitive real-time RT-PCR is reliably converted from mRNA to cDNA by reverse transcription with reproducible efficiency, because due to coamplification of cDNA from different MAGE genes drop-outs of single markers at the stage of reverse transcription may easily remain unrecognized in the PCR, e.g., by a positive signal derived from only one marker thus pretending successful detection of other presumably coamplified markers that indeed may have failed sufficient cDNA-synthesis although being expressed.

In another particularly preferred embodiment of the method of the present invention the PCR-primers for amplification of MAGE-cDNA comprise oligonucleotides selected from one of the following groups:

| | | SEQ |
|---|---|---|
| PCR-primer | sequence (5′-3′) | ID NO: |
| (C) | | |
| MAGE-A1 | GTA GAG TTC GGC CGA AGG AAC | 51 |
| MAGE-A1 | CAG GAG CTG GGC AAT GAA GAC | 52 |
| MAGE-A2 | CAT TGA AGG AGA AGA TCT GCC T | 53 |
| MAGE-A2 | GAG TAG AAG AGG AAG AAG CGG T | 54 |
| MAGE-A3/6 | GAA GCC GGC CCA GGC TCG | 55 |
| MAGE-A3/6 | GAT GAC TCT GGT CAG GGC AA | 56 |
| MAGE-A4 | CAC CAA GGA GAA GAT CTG CCT | 57 |
| MAGE-A4 | TCC TCA GTA GTA GGA GCC TGT | 58 |
| MAGE-A10 | CTA CAG ACA CAG TGG GTC GC | 59 |
| MAGE-A10 | GCT TGG TAT TAG AGG ATA GCA G | 60 |
| MAGE-A12 | TCC GTG AGG AGG CAA GGT TC | 61 |
| MAGE-A12 | ATC GGA TTG ACT CCA GAG AGT A | 62 |
| (D) | | |
| MAGE-A1 | TAG AGT TCG GCC GAA GGA AC | 63 |
| MAGE-A1 | CTG GGC AAT GAA GAC CCA CA | 64 |
| MAGE-A2 | CAT TGA AGG AGA AGA TCT GCC T | 65 |
| MAGE-A2 | CAG GCT TGC AGT GCT GAC TC | 66 |
| MAGE-A3/6 | GGC TCG GTG AGG AGG CAA G | 67 |
| MAGE-A3/6 | GAT GAC TCT GGT CAG GGC AA | 68 |
| MAGE-A4 | CAC CAA GGA GAA GAT CTG CCT | 69 |
| MAGE-A4 | CAG GCT TGC AGT GCT GAC TCT | 70 |
| MAGE-A10 | ATC TGA CAA GAG TCC AGG TTC | 71 |
| MAGE-A10 | CGC TGA CGC TTT GGA GCT C | 72 |
| MAGE-A12 | TCC GTG AGG AGG CAA GGT TC | 73 |
| MAGE-A12 | GAG CCT GCG CAC CCA CCA A | 74 |

This embodiment of the invention is advantageous because it is capable of measuring the individual expression of all those members of the MAGE-A subfamily encoding target antigens recognized by cytotoxic T lymphocytes, which are thus relevant for tumor vaccination. In the particular case of MAGE-A3 and 6, which are amplified by the same pairs of PCR-primers depicted in group C and D, there is no loss of information relevant for vaccine design caused by the coamplification, because the proteins encoded by MAGE-A3 and 6 are almost identical due to a sequence homology of 99%.

In an even more preferred embodiment of the method of the present invention primers of group C are used for a first round and/or primers of group D for a second round of PCR-amplification. This embodiment of the invention is advantageous for carrying out a highly sensitive nested or semi-nested real-time MAGE RT-PCR.

In another embodiment of the method of the present invention a single or double pair of PCR-primers is used amplifying all members of the selected group of MAGE genes, respectively. This embodiment relates to a highly sensitive real-time RT-PCR specifically detecting the expression of more than one MAGE gene, by a single pair of pan-MAGE PCR-primers in case of a single-step PCR or a double pair of pan-MAGE PCR-primers in case of a nested or semi-nested PCR. Due to the high level of sequence homology among the different MAGE genes, sites of sequence identity between all members of a selected group of MAGE genes may be found by computer-based sequence analysis, where such pan-MAGE PCR-primers can hybridize.

As with every pair of PCR-primers, either monospecific for the cDNA of an individual MAGE gene or oligospecific for the cDNAs of some or all members of a certain group of MAGE genes (=pan-MAGE PCR-primer), primer positions have to be selected in a way to avoid amplification of genomic MAGE DNA. For example, amplification of genomic MAGE-sequences can be avoided by the use of primers localized in different exons or primers spanning different neighboring exons, thus restricting hybridization to cDNA only. Furthermore, the positions of the PCR-primers have to be chosen to fall within the sequence segment(s) of the MAGE transcript(s), which is (are) reverse transcribed by the actual RT-primer(s) used for cDNA-synthesis.

Because of its quantitative nature, the highly sensitive real-time RT-PCR of the present invention is particularly useful for measuring the load of disseminated tumor cells in individual patients, thus estimating the probability of a metastatic relapse originating from the early tumor cell spread that took place prior to successful treatment of the primary tumor. Thus, the present invention provides a method that precisely predicts whether there is a requirement of an adjuvant tumor therapy by determining the MAGE-A gene expression level.

Accordingly, the quantification of MAGE-A gene expression level is based on calculating the calibrator normalized relative ratio, according to the following formula:

$$Normalized Ratio = \frac{\frac{{N}_{To ( S )}}{{N}_{Ro ( S )}}}{\frac{{N}_{To ( C )}}{{N}_{Ro ( C )}}} = \frac{\frac{{E}_{T}^{CpT ( C )}}{{E}_{R}^{CpR ( C )}}}{\frac{{E}_{T}^{CpT ( S )}}{{E}_{R}^{CpR ( S )}}} = \frac{{E}_{T}^{CpT ( C )}}{{E}_{R}^{CpR ( C )}} \times \frac{{E}_{R}^{CpR ( S )}}{{E}_{T}^{CpT ( S )}} = {E}_{T}^{CpT ( C ) - CpT ( S )} \times {E}_{R}^{CpR ( S ) - CpR ( C )}$$

N_{To}/N_{Ro}: Initial number of target/reference molecules, i.e., of MAGE cDNAs/reference cDNA
CpT: Cycle number at target detection threshold (crossing point)
CpR: Cycle number at reference detection threshold (crossing point)
E_{T}: Efficiency of target amplification
E_{R}: Efficiency of reference amplification
T: Target (i.e., MAGE gene(s))
R: Reference (i.e., reference genes)
S: Tissue sample of a tumor patient (i.e., patient sample)
C: Calibrator sample
   PCR amplification is described by the basic equation:

N=N₀×E^{Cp}
N: number of molecules at a certain cycle
N₀: initial number of molecules
E: amplification efficiency
Cp: cycle number at detection threshold (crossing point)
   The ratio of target to reference for each patient sample and for the calibrator sample is calculated in the first step. This corrects for sample to sample variations caused by differences in the initial quality and quantity of the nucleic acid.

The target/reference ratio of each patient sample is then divided by the target/reference ratio of the calibrator sample. This second step normalizes for different detection sensitivities of target and reference amplicons caused by different probe annealing, quantum yields of dye batches or FRET (fluorescence resonance energy transfer) efficiency.

The final ratio resulting from the calibrator normalized relative quantification is only a function of PCR-efficiency, and of the determined crossing points, i.e., the cycle number at detection threshold. Thus, the Cp-value represents the cycle at which PCR amplification begins its exponential phase and is considered the point that is most reliably proportional to the initial concentration. It does not require the knowledge of absolute copy numbers at the detection threshold. The PCR-efficiencies as well as the Cp-value of target and reference gene for patient sample and calibrator sample is obtained by the Light Cycler quantification analysis software (e.g., LightCycler Relative Quantification Software, Roche Molecular Biochemicals, Mannheim, Germany).

The calculation of this calibrator normalized ratio does not require a standard curve in each LightCycler analysis run. The calibrator sample is typically a positive sample with a stable ratio of the initial number of target molecules and reference molecules and is used to normalize all samples within one run, but in addition provides a constant calibration point between several LightCycler runs.

The relative expression level of a target gene and a reference gene is determined for each patient sample and one calibrator sample, integrated in each LightCycler run. Thus the normalization to a calibrator sample provides a constant calibrator point between PCR runs.

Values and intervals of MAGE-A gene expression levels (V) as disclosed herein are valid in connection with the use (comprising total amounts and dilutions) of calibrator samples based on cell lines Mz2-Mel and LB23-SAR (C) as specified by the protocols herein.

If calibrator samples (CE) other than those specified herein (C) are used, e.g., differing in cell numbers spiked for the preparation of calibrator samples and/or using other cell lines than Mz2-Mel and LB23-SAR the equivalents (VE) for the values and intervals of MAGE-A gene expression levels as disclosed herein (V) have to be calculated as follows:

$$\frac{\frac{{N}_{To ( S )}}{{N}_{Ro ( S )}}}{\frac{{N}_{To ( CE )}}{{N}_{Ro ( CE )}}} = \frac{\frac{{N}_{To ( S )}}{{N}_{Ro ( S )}}}{\frac{{N}_{To ( C )}}{{N}_{Ro ( C )}}} \times \frac{\frac{{N}_{To ( C )}}{{N}_{Ro ( C )}}}{\frac{{N}_{To ( CE )}}{{N}_{Ro ( CE )}}}$$

$$VE = V \times F$$

Thus, the equivalents (VE) of values and intervals of MAGE-A gene expression levels as disclosed herein (V) are obtained by multiplication with the conversion factor F. Conversion factor F for calibrator samples other than those specified herein can be determined as follows:

$$F = \frac{\frac{{N}_{To ( C )}}{{N}_{Ro ( C )}}}{\frac{{N}_{To ( CE} )}{{N}_{Ro ( CE )}}} = \frac{\frac{{E}_{T}^{CpT ( CE )}}{{E}_{R}^{CpR ( CE )}}}{\frac{{E}_{T}^{CpT ( C )}}{{E}_{R}^{CpR ( C )}}}$$

A MAGE-A gene expression level in blood or bone marrow at the time of tumor removal which is equal to or above a threshold value selected from the interval of 0.01 to 1.0, preferably selected from the interval of 0.05 to 0.5, more preferably selected from the interval of 0.1 to 0.3 and most preferably identical to 0.2 is a statistically significant and independent predictor of survival in solid tumor patients after tumor removal such as in resected NSCLC and most strongly correlates with the development of distant metastasis. Quantification of MAGE-A expression in bone marrow and blood by the multimarker real-time RT-PCR measures systemic MRD and predicts the distant metastasis-free survival in individual patients including those with resected NSCLC reliably enough with an impact on overall survival to help in decision making on adjuvant therapy (i.e., further anti-cancer treatment subsequent to tumor removal such as surgery).

### A. DEFINITIONS

The term “RT” or “cDNA synthesis” is used in the current invention for the conversion of mRNA into complementary DNA (cDNA) by a reverse transcriptase enzyme in a reverse transcription reaction (RT).

The term “RT-PCR” is used in the current invention for methods applying a polymerase chain reaction (PCR) after converison of mRNA into complementary DNA (cDNA) by a reverse transcription reaction (RT).

The term “conventional PCR” is used in the current invention for non-fluorescent PCR methods operated on all kinds of traditional thermocyclers.

The term “nested PCR” is used in the current invention for PCR methods comprising two amplification steps with different sets of primers for the first and second round of amplification.

The term “semi-nested PCR” is used in the current invention for PCR methods comprising two amplification steps with one shared primer for the first and second round of amplification.

The term “real-time PCR” is used in the current invention for fluorescence-based PCR methods on photometric thermocyclers with the option for quantification of original template amounts. The method can include additional preamplification steps on a traditional thermocycler for a defined number of PCR-cycles.

The term “multimarker MAGE PCR” is used in the current invention for PCR assays that enable the separate amplification of cDNA of different individual MAGE genes.

The term “pan MAGE PCR” is used in the current invention for PCR assays that enable the amplification of cDNA of different MAGE genes by one or more pairs of consenus PCR-primers each capable of coamplifying at least two different MAGE gene transcripts.

The term “RT-primer” or “cDNA synthesis primer” is used in the current invention for oligonucleotides designed to hybridize only to a defined target mRNA to yield specific cDNA molecules of these transcripts in a reverse transcription reaction.

The term “PCR primer” is used in the current invention for oligonucleotides designed to hybridize only to certain regions of target cDNA to yield amplicons of a specific length in a PCR reaction.

The term “high sensitivity” is used in the current invention for the capability of a PCR method to yield detectable MAGE specific amplificates from 5 or less tumor cells in 2 ml of whole blood. Additionally a crossing point below 30 PCR-cycles is required for real-time PCR-methods to fulfil the definition.

The term “solid tumor” as used in the present invention refers to malignant neoplasias such as carcinomas, sarcomas and lymphomas which unlike leukemia and myeloma form abnormal masses of tissue. In total solid tumors are much more frequent than leukemia and myeloma.

The term “primary tumor” as used in the present invention refers to the totality of tumor tissue undergoing tumor removal with curative intent, e.g., by surgery either at first tumor diagnosis or at diagnosis of tumor regrowth (i.e., relapse) after previous tumor removal.

### B. TECHNICAL REFERENCES

The following references, to the extent that they provide exemplary procedural or other details supplementary to those set forth herein, are specifically incorporated herein by reference.
Sambrook, J., Russel, D., W. (2001). “Molecular Cloning—A Laboratory Manual.” 3^{rd} edition 2001, Cold Spring Harbor Laboratory Press,
Roche Molecular Biochemicals (2000). “LightCycler Operator's Manual.” Version 3.5
Meuer, S., Wittwer, C., Nakagawara, K., I. (Eds.) (2001). “Rapid Cycle Real-Time PCR—Methods and Applications.” Springer Publishing
Dietmaier, W., Wittwer, C., Sivasubramanian, N. (Eds.) (2002). “Rapid Cycle Real-Time PCR—Methods and Applications—Genetics and Oncology.” Springer Publishing
world-wide-web at roche-applied-science.com/lightcycler-online
world-wide-web at appliedbiosystems.com/techsupport

### C. EXAMPLES

The following examples are included to demonstrate certain non-limiting aspects of the disclosure. It should be appreciated by those of skill in the art that the techniques disclosed in the examples which follow represent techniques discovered by the inventor to function well in the practice of the disclosure. However, those of skill in the art should, in light of the present disclosure, appreciate that many changes can be made in the specific embodiments which are disclosed and still obtain a like or similar result without departing from the spirit and scope of the disclosure.

### Example 1

### Materials and Methods

Trial Design.

Patients with suspected localized NSCLC (UICC stage Ia-IIIa) planned to undergo tumor resection were enrolled in a prospective clinical study. The study protocol was approved by the responsible ethics committee and all patients gave written informed consent to participate in the study. Preoperative staging included computed tomography of the head, chest, and abdomen as well as a bone scintigraphy. Patients with overt distant metastasis, with neoadjuvant therapy or a history of further malignant disease were excluded.

In general, a lobectomy or pneumonectomy with systematic mediastinal lymphadenectomy was performed. No adjuvant chemotherapy was given, as it was not established as standard of care in the study interval. After resection all tumors were classified according to the WHO classification for histologic tumor typing¹⁵. The tumor stage was classified according to the 6^{th} edition of the International Union against Cancer (UICC) tumor-node-metastasis classification (TNM6)¹⁶. Only those patients with histologically confirmed NSCLC and complete tumor resection were finally included in the prospective study.

Follow-up assessments comprised physical examination, chest X-ray, and blood tests at 3-month interval and an additional thoracic computed tomography scan, abdominal ultrasound, and bronchoscopy at 6-month interval. In addition, family practitioners were contacted to obtain information about locoregional relapse, distant metastasis, and death. The median observation period was 43 months (range 1-95 months).

The primary endpoint of the study was postoperative distant-metastasis-free survival. Distant-metastasis-free survival was defined as the postoperative time to distant metastasis without prior locoregional recurrence. Secondary endpoints were cancer-free survival and overall survival. Cancer-free survival was defined as the postoperative time to any locoregional recurrence or distant metastasis. Overall survival was defined as postoperative time to death from any cause. Locoregional-recurrence-free survival was defined as the postoperative time to locoregional recurrence without prior distant metastasis.

Clinical Samples and Preanalytical Preparation.

All patients underwent bilateral bone marrow aspiration through an aspiration needle from each anterior iliac crest and donated peripheral blood at the time of primary operation immediately before thoracotomy. One mL of native bone marrow and 2 mL of blood were directly mixed with 4 and 8 mL, respectively, of denaturating nucleic acid extraction buffer [4 M guanidine isothiocyanate, 0.5% sarcosyl (N-laurylsarcosine sodium salt), 25 mM sodium citrate (pH=7.0), 0.7% 2-mercaptoethanol] to ensure immediate lysis of the probe providing full RNA protection. Samples were stored at −20° C. until further preparation within 4 weeks.

Quantitative Multimarker MAGE Real-Time RT-PCR.

Unless stated otherwise herein, the multimarker MAGE real-time RT-PCR method including primer sequences and reaction conditions was performed as described in KUFER P, et al, CANCER RES 2002; 62(1): 251-61⁵ and MECKLENBURG I, et al., J IMMUNOL METHODS 2007; 323(2): 180-93¹⁴.

Preparation of Clinical Samples.

RNA preparation and cDNA synthesis from clinical samples: Total RNA from all clinical samples was isolated by phenol/chloroform extraction and resuspended in 50 μL of DEPC-treated water. 10 μL of total RNA were used in the subsequent cDNA synthesis using the Omniscript RT Kit (Qiagen, Hilden, Germany) in 20 μL total reaction volume with 2 μL of 10× Buffer RT, 2 μL of the supplied dNTP mix, 50 pmol of each specific reverse-transcription primer pan-MAGE-RT [5′-ACC TGC CGG TAC TCC AGG-3′, SEQ ID NO: 75] and PBGD-RT [5′-TGG GGC CCT CGT GGA ATG-3′, SEQ ID NO: 76], 10 units of RNAse inhibitor (Roche Molecular Biochemicals, Mannheim, Germany) and 4 units of Omniscript RT enzyme. The reverse transcription (RT) reaction was carried out in a GeneAmp PCR System 9700 (Applied Biosystems, Foster City, USA) for 60 min at 37° C. followed by denaturation at 93° C. for 5 min.

Preamplification of MAGE-A from cDNA of Clinical Samples:

Two μL of the cDNA were used for the first round of PCR in 20 μL reactions with 2 μL of 10×PCR Buffer (200 mM Tris, pH=8.0, 500 mM KCl), 1.5 μM MgCl₂, 0.2 μM of each dNTP, 1 unit Platinum Taq DNA polymerase (all by Invitrogen, Groningen, Netherlands) and 0.2 μM of each outer MAGE primer in a GeneAmp PCR System 9700 (Applied Biosystems, Foster City, USA) according to the following cycle profile: enzyme activation at 95° C. for 3 min; denaturation at 95° C. for 30 s, annealing at 60° C. for 45 s and extension at 72° C. for 1 min for 20 cycles followed by terminal extension at 72° C. for 7 min.

Preparation of Calibrator Samples.

Calibrator samples for MAGE-A1, -A2, -A3/6, -A10 and -A12 consist of two mL of whole blood from a healthy human donor spiked with 10 tumor cells of the cultured human melanoma cell line Mz2-Mel.

Calibrator samples for MAGE-A4 consist of two mL of whole blood from a healthy human donor spiked with 10 tumor cells of the cultured human sarcoma cell line LB23-SAR. Each 2 mL-calibrator sample was directly mixed with 8 mL of denaturating nucleic acid extraction buffer [4 M guanidine isothiocyanate, 0.5% sarcosyl (N-laurylsarcosine sodium salt), 25 mM sodium citrate (pH=7.0), 0.7% 2-mercaptoethanol].

Total RNA from each 2 mL-calibrator sample was isolated by phenol/chloroform extraction as for the clinical blood- and bone marrow samples from tumor patients and resuspended in 20 μL of DEPC-treated water. Total RNA from 5 such calibrator samples for MAGE-A1, -A2, -A3/6, -A10 and -A12 and from 5 such calibrator samples for MAGE-A4 were pooled, respectively, resulting in 5×20 μL=100 μL of calibrator pool RNA. RNA is stored at −80° C.

Calibrator cDNA for MAGE-A1, -A2, -A3/6, -A10 and -A12 and calibrator cDNA for MAGE-A4 was generated like cDNA for the clinical blood- and bone marrow samples from tumor patients except that 5 μL instead of 12 μL RNA were used:
7.75 μL DEPC-H₂O
2 μL Buffer RT (10×)
2 μL dNTP Mix (5 mM)
1 μL pan-MAGE-RT Primer (50 pmol/μL)
1 μL PBGD-RT Primer (50 pmol/μL)
0.25 μL RNAse Inhibitor (40 U/μL, Roche)
1 μL Omniscript RT
5 μL calibrator pool RNA
20 μL total volume
   cDNA from 4 such independent cDNA syntheses for MAGE-A1, -A2, -A3/6, -A10 and -A12 and from 4 such independent cDNA syntheses for MAGE-A4 are pooled, respectively, resulting in 4×20 μL=80 μL of calibrator pool cDNA.

Preamplification of MAGE-A from Calibrator Pool cDNA:

Two μL of calibrator pool cDNA for MAGE-A1, -A2, -A3/6, -A10 and -A12 or 2 μL of calibrator pool cDNA for MAGE-A4 were used for the preamplification-PCR in 50 μL reactions with 5 μL of 10×PCR Buffer (200 mM Tris, pH=8.0, 500 mM KCl), 1.5 μM MgCl₂, 0.2 μM of each dNTP, 1 unit Platinum Taq DNA polymerase (all by Invitrogen, Groningen, Netherlands) and 0.2 μM of the respective outer MAGE primer pair in a GeneAmp PCR System 9700 (Applied Biosystems, Foster City, USA) according to the following cycle profile: enzyme activation at 95° C. for 3 min; denaturation at 95° C. for 30 s, annealing at 60° C. for 45 s and extension at 72° C. for 1 min for 20 cycles followed by terminal extension at 72° C. for 7 min.

Preamplified MAGE-A1, -A2, -A3/6, -A10 and -A12 as well as preamplified MAGE-A4 from 4 such independent preamplification runs were pooled, respectively, resulting in 4×50 μL=200 μL of preamplified MAGE-A calibrator pool (i.e., 200 μL of MAGE-A1, 200 μL of MAGE-A2, 200 μL of MAGE-A3/6, 200 μL of MAGE-A10, 200 μL of MAGE-A12 and 200 μL of MAGE-A4). Each preamplified MAGE-A calibrator pool was diluted 1:10 with H₂O (i.e., 900 μL H₂O plus 100 μL preamplifed MAGE-A1, -A2, -A3/6, -A10, -A12 or -A4 calibrator pool) for use as MAGE calibrator probe in the Real-time PCR.

PBGD calibrator probes were prepared by diluting 36 μL of calibrator pool cDNA for MAGE-A1, -A2, -A3/6, -A10 and -A12 (derived from Mz2-Mel) and 36 μL of calibrator pool cDNA for MAGE-A4 (derived from LB23-SAR) with 324 μL H₂O, respectively, resulting in 360 μL of PBGD calibrator probe for use in the Real-time PCR of MAGE-A1, -A2, -A3/6, -A10 and -A12 and 360 μL of PBGD calibrator probe for use in the Real-time PCR of MAGE-A4

Real-Time PCR.

For real-time PCR we prepared 15 μL reactions in LightCycler capillaries with 2 μL of the MAGE-A1, -A2, -A3/6, -A10, -A12 and -A4 preamplification reaction, respectively, 1.5 μL of FastStart DNA Master SYBR Green I reagent (Roche Molecular Biochemicals, Mannheim, Germany), 10 pmol of each inner MAGE primer in a final concentration of 2.5 μM MgCl₂ (for MAGE-A1, -A3/6 and -A4) or 3.0 μM MgCl₂ (for MAGE-A2, -A10 and -A12), respectively. Likewise, 15 μL reactions in LightCycler capillaries were prepared with 2 μL of MAGE-A1, -A2, -A3/6, -A10, -A12 and -A4 calibrator probes, respectively. The real-time PCR was run on a LightCycler 1.0 for 5 min at 95° C. for initial activation of the enzyme, 10 sec at 95° C. for denaturation, 5 sec at 60° C. for annealing and 10 sec at 72° C. for elongation for 40 cycles. After completion of the reaction the PCR products were subjected to a melting curve analysis spanning the temperature range of 65° C. to 95° C. with a ramping rate of 0.1° C./sec.

The housekeeping gene porphobilinogen desaminase (PBGD) was amplified from cDNA of the clinical samples in a separate real-time PCR using hybridization probes in 20 reactions with 1 μL of cDNA and 5 mM MgCl₂, 0.5 μM of sense primer [5′-AGA GTG ATT CGC GTG GGT ACC-3′, SEQ ID NO: 77], 0.5 μM of antisense primer [5′-TTG GGT GAA AGA CAA CAG CAT C-3′, SEQ ID NO: 78], 0.2 μM donor probe [5′-AGT GGA CCT GGT TGT TCA CTC CTT GAA-3′-Fluo, SEQ ID NO: 79], 0.2 μM acceptor probe [LCRed-640-5′-ACC TGC CCA CTG TGC TTC CTC CT-3′, SEQ ID NO: 80] and 2 μL of FastStart DNA Master Hybridization probes reaction mix (Roche Molecular Biochemicals, Mannheim, Germany). Likewise, 20 μL reactions in LightCycler capillaries were prepared with 1 μL of PBGD calibrator probes derived from Mz2-Mel and LB23-SAR, respectively. The real-time PCR on LightCycler 1.0 was run as follows: initial enzyme activation for 5 min at 95° C., denaturation for 15 sec at 95° C., annealing at 60° C. for 10 sec and extension for 20 sec at 72° C.

Calculation of MAGE-A Gene Expression Levels.

MAGE-A gene expression levels were calculated as calibrator normalized relative ratio, which is the relative amount target/reference (i.e., preamplified MAGE-A/non-preamplified PBGD) determined from each clinical sample [N_{To(S)}/N_{Ro(S)}] divided through the ratio target/reference (i.e., preamplified MAGE-A/non-preamplified PBGD) determined from the calibrator [N_{To(C)}/N_{Ro(C)}]:

$$Normalized Ratio = \frac{\frac{{N}_{To ( S )}}{{N}_{Ro ( S )}}}{\frac{{N}_{To ( C )}}{{N}_{Ro ( C )}}} = \frac{\frac{{E}_{T}^{CpT ( C )}}{{E}_{R}^{CpR ( C )}}}{\frac{{E}_{T}^{CpT ( S )}}{{E}_{R}^{CpR ( S )}}} = \frac{{E}_{T}^{CpT ( C )}}{{E}_{R}^{CpR ( C )}} \times \frac{{E}_{R}^{CpR ( S )}}{{E}_{T}^{CpT ( S )}} = {E}_{T}^{CpT ( C ) - CpT ( S )} \times {E}_{R}^{CpR ( S ) - CpR ( C )}$$

### Abbreviations:

T: Target (=MAGE-A1, -A2, -A3/6, -A10, -A12 or -A4)
R: Reference (=PBGD)
S: Clinical Sample
C: Calibrator Sample
N_{To(S)}/N_{Ro(S)}: Number of target molecules (i.e., MAGE-A) determined from clinical sample after preamplification but prior to real-time PCR/Number of reference molecules (i.e., PBGD) determined from clinical sample without preamplification prior to real-time PCR
N_{To(C)}/N_{Ro(C)}: Number of target molecules (i.e., MAGE-A) determined from MAGE-A calibrator probe after preamplification but prior to real-time PCR/Number of reference molecules (i.e., PBGD) determined from PBGD calibrator probe without preamplification prior to real-time PCR
CpT: Cycle number at target detection threshold (crossing point)
CpR: Cycle number at reference detection threshold (crossing point)
E_{T}: Efficiency of target amplification
E_{R}: Efficiency of reference amplification
   Efficiencies E_{T} and E_{R} of target (i.e., MAGE-A) and reference (i.e., PBGD) amplification in the real-time PCR were calculated by means of serial calibrator dilutions covering several orders of magnitude¹⁴. Real-time PCR for MAGE-A as described herein was run with 2 μL undiluted preamplified MAGE-A calibrator pool and with 2 μL each of preamplified MAGE-A calibrator pool diluted 1:10, 1:100, 1:1000 and 1:5000 equivalent to 2×10⁻¹, 2×10⁻², 2×10⁻³, and 4×10⁻⁴ μL of undiluted preamplified MAGE-A calibrator pool, respectively. Real-time PCR for PBGD as described herein was run with 1 μL each of calibrator pool cDNA diluted 1:10, 1:100, 1:1000 and 1:5000 equivalent to 2×10⁻¹, 2×10⁻², 2×10⁻³, and 4×10⁻⁴ μL of calibrator pool cDNA, respectively. The resulting standard curves were exported to the LightCycler relative quantification software and were used as coefficient files to correct for differences in PCR efficiencies.

The calculation of the calibrator normalized relative ratios (i.e., MAGE-A gene expression levels) was carried out in an automated manner using a specific calculation software (e.g., LightCycler Relative Quantification Software, Roche Molecular Biochemicals, Mannheim, Germany). The lower limit of quantification (=LLOQ) was 0.01.

Under consideration of all dilution and preamplification factors the MAGE-A gene expression level as determined herein is equal to 2.5-times the number of MAGE-A mRNA molecules per PBGD mRNA molecules in the blood or bone marrow sample from a cancer patient relative to/divided through the number of MAGE-A mRNA molecules per PBGD mRNA molecules in the calibrator sample consisting of 2 mL of healthy blood spiked with 10 Mz2-Mel melanoma cells or LB23-SAR sarcoma cells.

### Example 2

### Results

Characteristics of the Patients.

In total 116 patients with suspected lung cancer without neoadjuvant therapy were enrolled in the study. According to postoperative assessment, 94 patients with histopathologically confirmed non-small cell lung cancer (NSCLC) fulfilled the inclusion criteria. In total, 22 patients dropped out because of a benign histology such as tuberculosis or pneumonia (n=5), small-cell lung cancer (n=4) or incomplete tumor resection (n=13). Among the 94 patients with NSCLC included in the study, clinicopathological characteristics were as shown in Table 2. Patients were all free of distant metastases. 42 had squamous cell carcinoma, 37 adenocarcinoma, 8 adenosquamous carcinoma and 7 were diagnosed with large cell carcinoma. The median age at the time of surgery was 66 years (range, 44-82 years). Distribution of histopathological grading was G1 in 3 patients, G2 in 37 patients, G3 in 53 patients, and G4 in 1 patient. Distribution of tumor size was pT₁ in 21 patients, pT₂ in 43 patients, pT₃ in 18 patients, and pT₄ in 12 patients. Distribution of lymph node status was pN₀ in 52 patients (55.3%), pN₁ in 20 patients (21.3%), and pN₂ in 22 patients (23.4%). During tumor surgery, ipsilateral intrapulmonary secondary lesions were found in 9 patients, which could be removed in parallel.

Of these 94 patients, follow-up information was available for 89 (94.7%): The median observation period in the study was 43 months (range 1-95 months). As shown in Table 3, 49 patients died within the observation period (55.1%): 36 patients died from cancer (40.5%), in 13 cases the death was unrelated to the malignant disease. Tumor recurrences were diagnosed in 40 patients (44.9%). 30 patients developed distant metastasis (33.7%): In 24 patients, distant metastasis occurred as first relapse event (27.0%), in six cases distant metastasis developed after locoregional relapse. 16 patients developed locoregional tumor recurrence (18.0%), which occurred as first relapse event in all cases.

Out of 94 NSCLC patients included in this prospective clinical study 41 presented with quantifiable MAGE-A expression in bone marrow or blood (43.6%). Log-rank tests revealed a significant decrease in overall survival (p=0.007), cancer-free survival (p=0.002), and distant-metastasis-free survival (p<0.001) of patients with MAGE-A expression ≧0.2 in at least one sample of bone marrow or blood compared to patients with MAGE-A expression levels below 0.2 in all samples. The corresponding hazard ratios (≧0.2 vs. <0.2) for death, cancer-related death and development of distant metastasis were 2.56 (95% CI, 1.42-4.63), 3.32 (95% CI, 1.66-6.61), and 4.03 (95% CI, 1.77-9.18), respectively. Five-year Kaplan-Meier estimates of distant-metastasis-free survival were 43% (MAGE-A expression level ≧0.2) versus 87% (MAGE-A expression level below 0.2). However, no statistically significant difference in locoregional-recurrence-free survival was found between these two patient subgroups.

MAGE-A Expression in Blood and Bone Marrow.

264 blood and bone marrow samples of the 94 patients with NSCLC were analyzed for MAGE-A gene expression by quantitative multimarker MAGE real-time RT-PCR. 15 samples dropped out because of vial damage during transportation and measurements of 3 samples were censored because of failure of amplification of the housekeeping marker PBGD.

In total, 1848 expression profiles of seven MAGE-A genes in 264 samples of 94 patients were created. The quantity of MAGE-A expression varied between the lower limit of quantification (LLOQ, i.e., 0.01) and >1,000.00. The mean MAGE/PBGD ratio was 67.8 in all cases at or above the LLOQ. Table 4 shows examples of the quantitative MAGE-A expression profiles of 11 selected patients. Values below the LLOQ are shown as negative.

Expression at or above the LLOQ of at least one MAGE-A gene in at least one sample of bone marrow or blood was detected in 43.6% of patients (n=41). Most frequently detected at or above the LLOQ was expression of MAGE-A1 in 8.0% of samples followed by MAGE-A2 (5.3%), MAGE-A12 (2.7%), MAGE-A3/6 (1.9%), MAGE-A10 (1.5%) and MAGE-A4 (1.5%). 13 patients showed quantifiable expression of more than one MAGE-A gene. 29 patients (31.2%) were exclusively MAGE-A positive in bone marrow, while 10 patients (11.9%) were exclusively MAGE-A positive in blood; 7 patients were MAGE-A positive in both bone marrow samples. Two patients were double positive in blood and in bone marrow.

No statistical correlations were found between quantifiable MAGE-A expression and tumor extension, grading, histology, lymph node status or age of the patients (Table 2).

Correlation of MAGE-A Expression with Survival.

In order to determine the impact of different MAGE-A expression levels in bone marrow or blood on patients' clinical outcome according to the primary endpoint, the distant-metastasis-free survival of patients with a MAGE-A expression level at or above a certain threshold value in at least one sample of bone marrow or blood was compared with patients with sub-threshold MAGE-A expression in all samples.

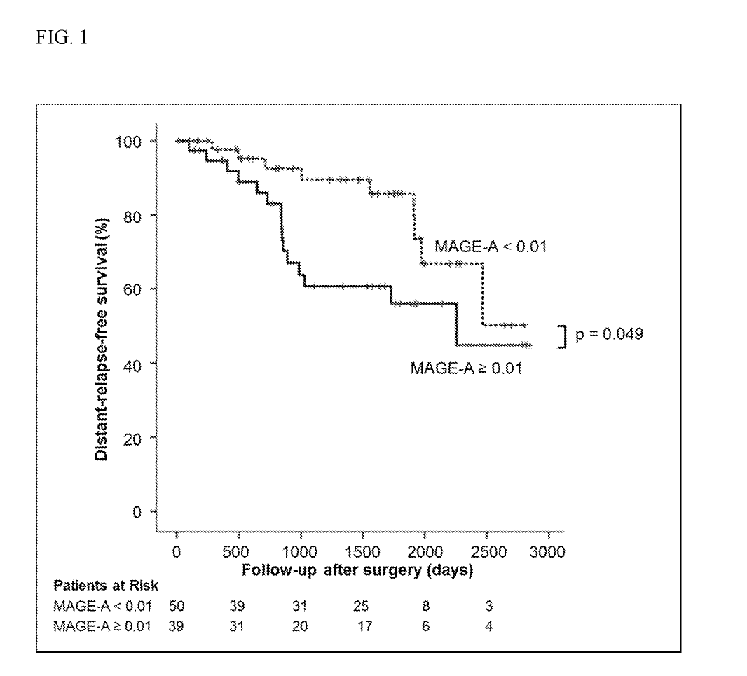

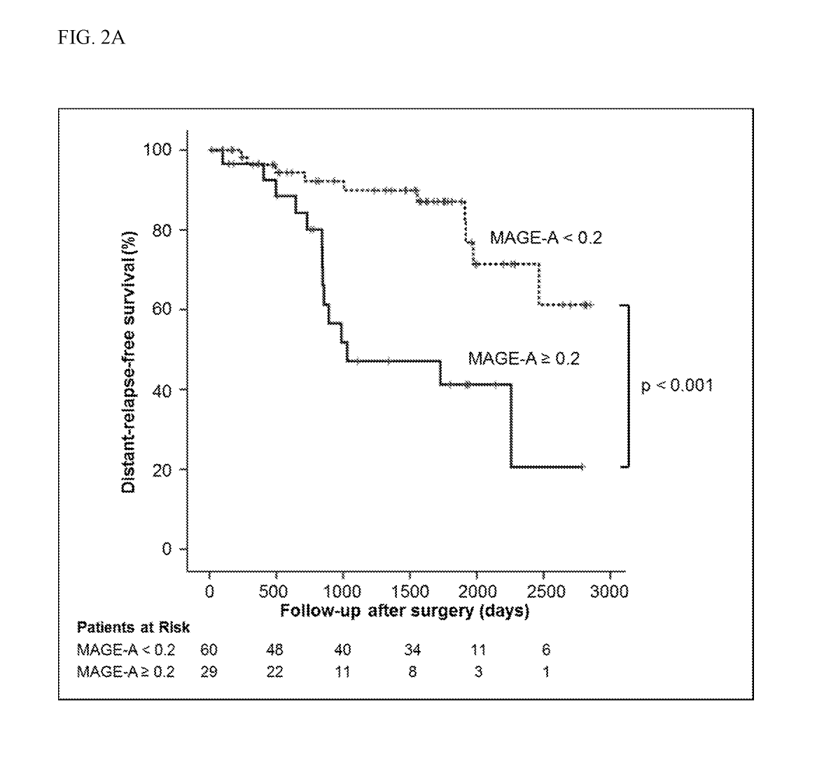

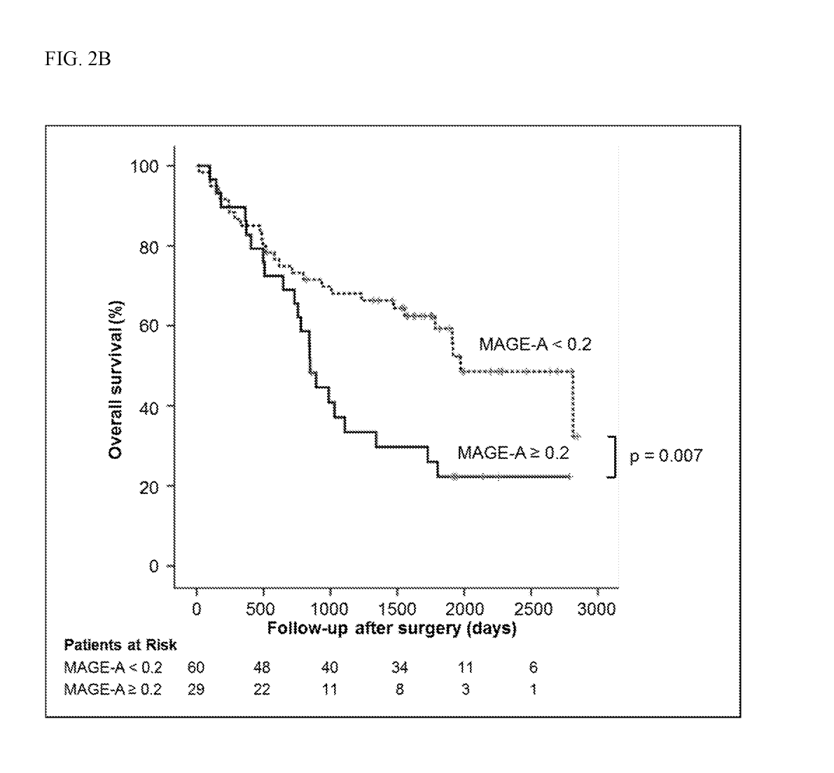

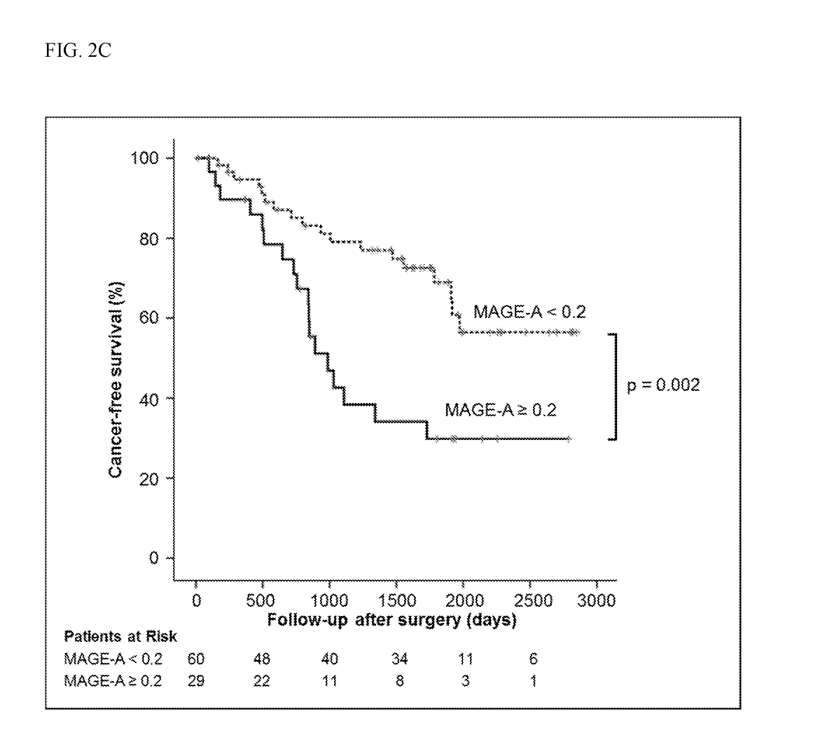

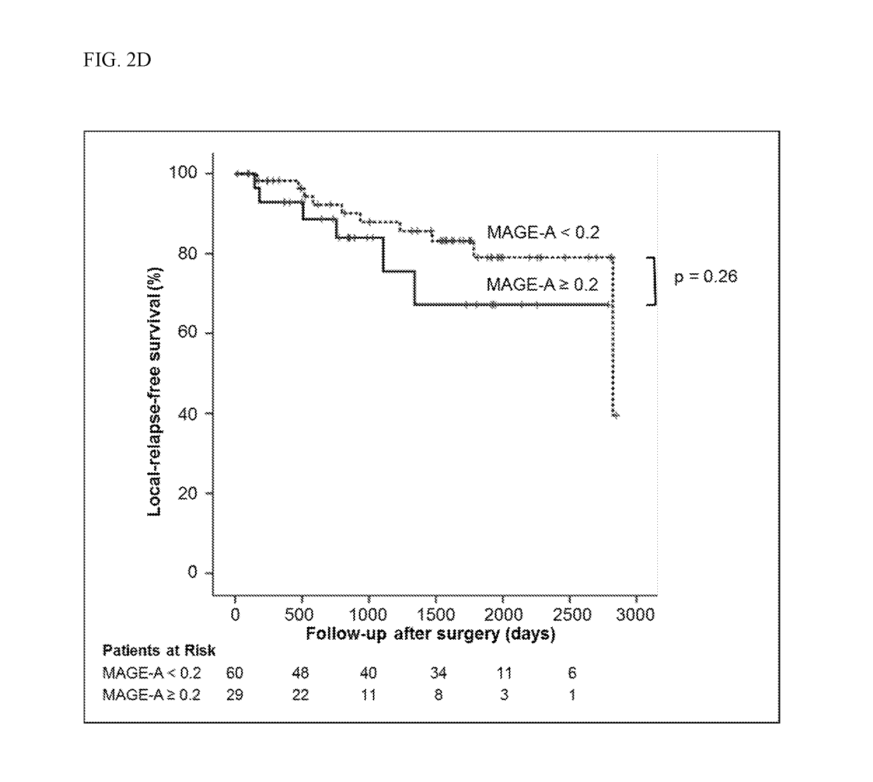

When patients with a MAGE-A expression level at or above the LLOQ (i.e., 0.01) in at least one sample of bone marrow or blood were compared with patients without quantifiable MAGE-A expression in all samples, the log-rank test revealed that the two patient subgroups differed with statistical significance (p=0.049, Table 5, FIG. 1). With a p-value cut-off at 10% even a MAGE-A expression level at or above 1.0 in at least one sample of bone marrow or blood versus below 1.0 in all samples showed some statistical significance (p=0.09, Table 5). However, when patients with a MAGE-A expression level at or above 0.05, 0.1, 0.2, 0.3 and 0.5 in at least one sample of bone marrow or blood were compared with patients below the respective MAGE-A expression threshold in all samples, the log-rank test revealed a statistically significant difference in distant-metastasis-free survival between the two patient subgroups with p-values of 0.013, 0.002, <0.001, 0.004 and 0.013, respectively (Table 5). Thus, with a p-value of <0.001 the MAGE-A expression level of ≧0.2 in at least one sample of bone marrow or blood versus MAGE expression below 0.2 in all samples clearly distinguishes best between patients with a higher risk of developing distant metastasis versus patients with a lower risk (FIG. 2A). The corresponding 5-year Kaplan-Meier estimates of distant-metastasis-free survival were 87% (95% CI±10%) for patients with MAGE-A expression <0.2 versus 43% (95% CI±11%) for ≧0.2.

As to the secondary endpoints, log-rank tests revealed a significant decrease in overall survival (p=0.007) and cancer-free survival (p=0.002) of patients with MAGE-A expression in at least one sample of bone marrow or blood at levels ≧0.2 compared to patients with MAGE-A expression levels below 0.2 in all samples (FIGS. 2b and 2c). The corresponding 5-year Kaplan-Meier estimates of overall survival were 59% (95% CI±14%) for patients with MAGE-A expression <0.2 versus 26% (95% CI±8%) for ≧0.2. The corresponding estimates for cancer-free survival were 69% (95% CI±14%) versus 31% (95% CI±18%).

In contrast to the strong correlation of MAGE-A expression at levels ≧0.2 in bone marrow or blood with the development of distant metastasis, there was no significant difference in locoregional-recurrence-free survival (p=0.26, log-rank test) between patients with MAGE-A expression levels ≧0.2 in bone marrow or blood and patients with sub-0.2 levels in all samples (FIG. 2D). Treatment failures according to MAGE-A expression at or above versus below threshold level 0.2 in bone marrow or blood and site of recurrence are shown in Table 3.

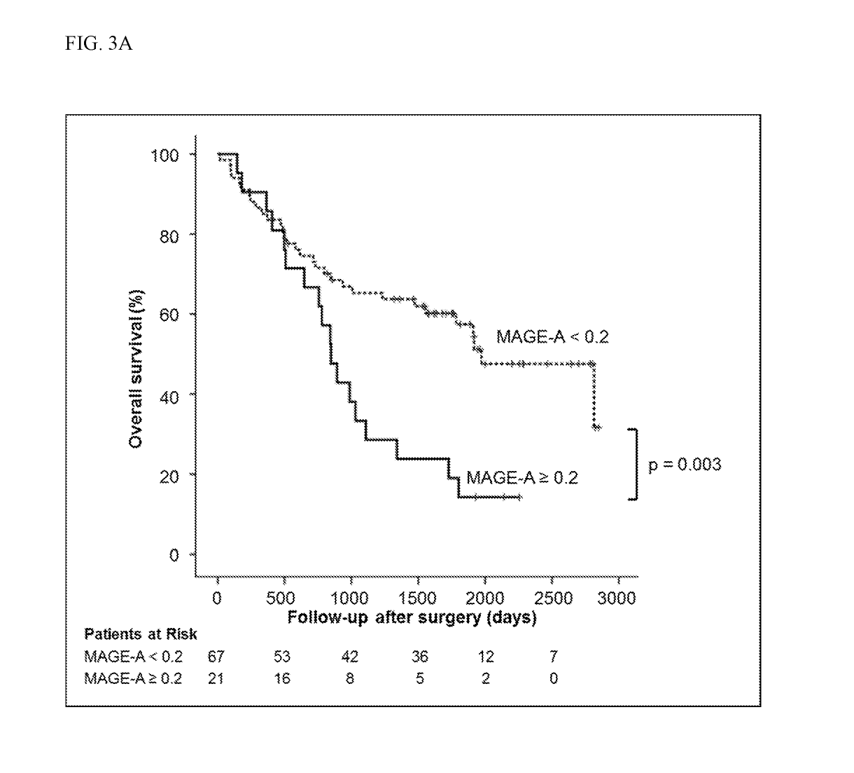

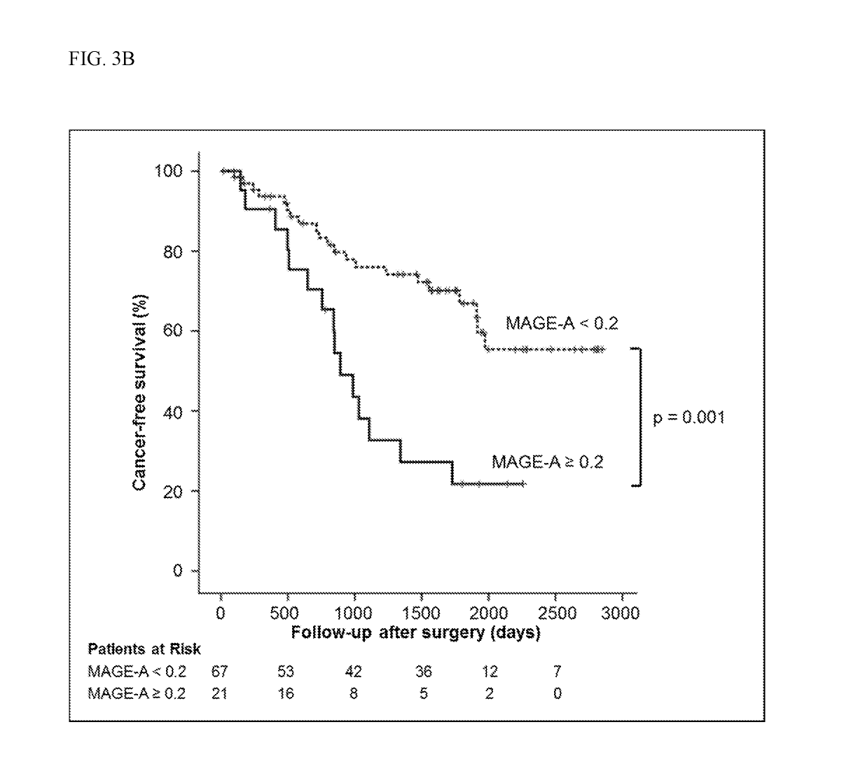

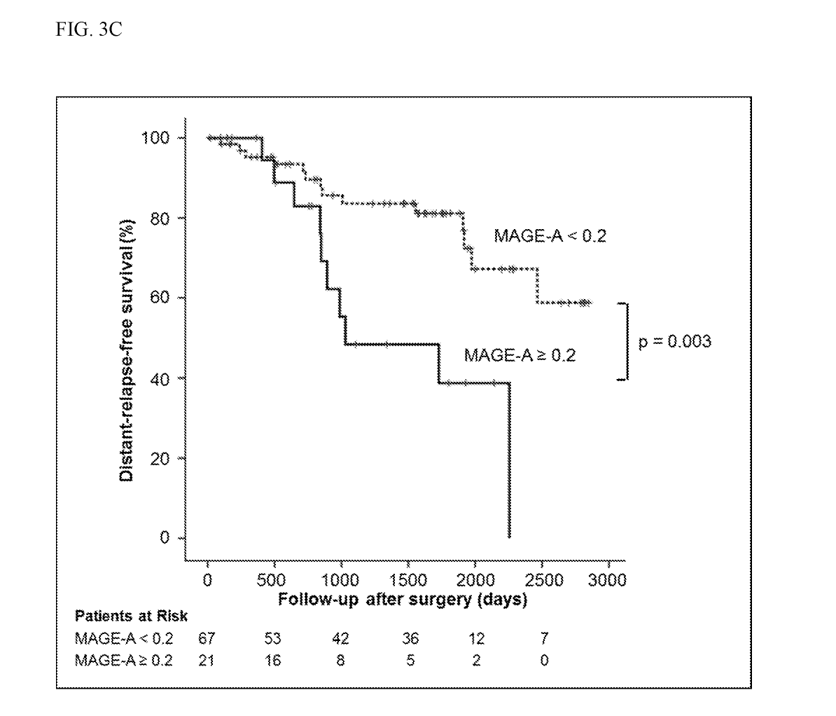

Further subgroup analyses addressed the impact of MAGE-A expression in bone marrow only versus MAGE-A expression in blood only using the 0.2 expression level threshold: Log-rank tests showed a significant decrease in overall survival (p=0.003, FIG. 3A), cancer-free survival (p=0.001, FIG. 3B), and distant-metastasis-free survival (p=0.003, FIG. 3C) in patients with MAGE-A expression at a level of ≧0.2 in at least one sample of bone marrow compared to patients with MAGE-A expression levels below 0.2 in both bone marrow samples. Because of the low number of patients with a quantifiable MAGE-A expression only in blood at or above a certain threshold no statistical significance could be reached in the blood-only analysis. However, the log-rank tests revealed that the p-values for the difference in distant-metastasis-free survival between the two patient subgroups (below versus at or above threshold) were minimal at the 0.2 MAGE-A expression threshold for both the blood-only (p=0.20) and the bone-marrow-only analysis (p=0.003). This confirmed the 0.2 MAGE-A expression level already found in the blood-and-bone marrow analysis as optimal threshold that distinguishes best between patients with a higher risk of developing distant metastasis and those with a lower risk.

MAGE-A Expression in Bone Marrow or Blood as Independent Predictor of Survival.

Multivariate analysis was used to test whether MAGE-A expression in bone marrow or blood is independent in its prognostic value for survival from other prognostic factors. This analysis was based on the 0.2 threshold for the MAGE-A expression level. A preceding univariate analysis had shown that beside the MAGE-A expression level in bone marrow or blood also tumor size and lymph node status were significant prognostic variables for overall and cancer-free survival (Table 6). Multivariate analysis using the Cox proportional-hazard model revealed that MAGE-A expression in bone marrow or blood at levels ≧0.2 is a significant prognostic factor predicting death of any cause (p=0.002), cancer-related death (p=0.001), and development of distant metastasis (p=0.001) independently from standard prognostic factors of survival such as tumor extension, tumor histology, grading, and age of patient at the time of surgery. MAGE-A expression at levels ≧0.2 in blood or bone marrow was the only significant predictor of distant metastasis with a hazard ratio (≧0.2 vs. <0.2) of 4.03 (95% CI, 1.77-9.18). Hazard ratios for cancer-related death were 3.32 (95% CI, 1.66-6.61 95% CI) for MAGE-A expression in blood or bone marrow (≧0.2 vs. <0.2), 1.65 (95% CI, 1.11-2.45) for tumor size (T₃₋₄ vs. T₁₋₂) and 1.56 (95% CI, 1.07-2.29) for lymph node status (N₂ vs. N₀₋₁). Hazard ratios for death of any cause were 2.56 (95% CI, 1.42-4.63) for MAGE-A expression in blood or bone marrow (≧0.2 vs. <0.2), 1.71 (95% CI, 1.23-2.38) for tumor size (T₃₋₄ vs. T₁₋₂) and 1.39 (95% CI, 1.00-1.93) for lymph node status (N₂ vs. N₀₋₁).

This prospective study with a median observation period of 43 months demonstrated that MAGE-A expression at levels ≧0.2 in blood or bone marrow at the time of tumor surgery is a strong and independent predictor of survival in patients with resected non-small cell lung cancer.

The decreases in overall survival, cancer-free survival, and distant metastasis-free survival of patients with MAGE-A expression in at least one sample of bone marrow or blood at levels ≧0.2 compared to patients with MAGE-A expression levels <0.2 in all samples were highly significant by univariate analysis with p-values of 0.007, 0.002 and <0.001, respectively. The corresponding 5-year Kaplan-Meier estimates of overall survival, cancer-free survival, and distant-metastasis-free survival for patients with MAGE-A expression <0.2 were 59% (95% CI±14%), 69% (95% CI±14%) and 87% (95% CI±10%), respectively. As MAGE-A expression in blood or bone marrow has the largest impact on distant metastasis-free survival compared to cancer-free and overall survival both in terms of the best p-value in univariate analysis and of the best 5-year Kaplan-Meier estimate—87% of patients with MAGE-A expression <0.2 stay free of distant metastasis—quantification of MAGE-A expression in bone marrow or blood indeed seems to measure systemic MRD that frequently grows out to form distant metastases. Further support for this conclusion comes from the observation that MAGE-A expression in blood or bone marrow does not seem to have an impact on the development of local recurrences as indicated by the lack of a significant difference in locoregional-recurrence-free survival between patients with MAGE-A expression in at least one sample of bone marrow or blood at levels ≧0.2 compared to patients with MAGE-A expression levels <0.2 in all samples.

As to the impact on survival of MAGE-A expression in bone marrow versus MAGE-A expression in blood, the results of this study indicate that MAGE-A expression at levels ≧0.2 in bone marrow as well as MAGE-A expression at levels ≧0.2 in blood both contribute to the prediction of an unfavorable clinical outcome. Although the blood-only analysis itself did not reach statistical significance because of the low number of patients with a quantifiable MAGE-A expression only in blood, the p-value for the decrease of distant-metastasis-free survival in patients with MAGE-A expression at a level of ≧0.2 in at least one sample compared to patients with MAGE-A expression levels below 0.2 in all samples further improved in the log-rank test when MAGE-A expression in blood was included (p<0.001) compared to the bone marrow-only analysis (p=0.003).

Multivariate analysis using the Cox proportional-hazard model confirmed that MAGE-A expression at levels ≧0.2 in blood or bone marrow is indeed a highly significant predictor of an unfavorable outcome i.e., death of any cause (p=0.002), cancer-related death (p=0.001), and development of distant metastasis (p=0.001). In addition, the multivariate analysis demonstrated, that MAGE-A expression in bone marrow or blood is independent in its prognostic value for survival from other prognostic factors. The corresponding hazard ratios revealed that patients with MAGE-A expression levels ≧0.2 in at least one sample of bone marrow or blood had a 2.56-, 3.32-, and 4.03-fold increased risk of death, cancer-related death, and development of distant metastasis, respectively, compared to patients with MAGE-A expression levels below 0.2 in all samples. For comparison, the risks related to tumor size (T3-4 vs. T1-2) for death and cancer-related death were increased 1.71- and 1.65-fold and the risks related to lymph node status (N2 vs. N0-1) for death and cancer-related death were increased 1.39- and 1.56-fold, respectively. Thus, MAGE-A expression at levels ≧0.2 in blood or bone marrow was associated with the highest increase in relative risk for death of any cause and cancer-related death compared to tumor size (T3-4 vs. T1-2) and lymph node status (N2 vs. NO-1). Moreover, MAGE-A expression at levels ≧0.2 in blood or bone marrow was the only significant predictor of distant metastasis, for which it is associated with the highest increase in relative risk (4.03-fold) compared to cancer-related death (3.32-fold) and death of any cause (2.56-fold). This again underlines that quantification of MAGE-A expression in bone marrow and blood indeed measures systemic MRD that frequently grows out to form distant metastases.

In summary, quantification of MAGE-A expression in bone marrow and blood by the multimarker real-time RT-PCR assay for transcript amplification of MAGE-A1, -A2, -A3/6, -A4, -A10 and -A12 as used in this prospective study¹⁴ predicts the distant metastasis-free survival in individual patients with resected NSCLC reliably enough with a statistically proven impact on overall survival to help in decision making on adjuvant therapy. Thus, the potential impact of MAGE-A based MRD assessment in resected NSCLC compares to MRD assessment in B-lineage acute lymphoblastic leukemia (B-ALL), where it has advanced from an explorative method into an established staging procedure for clinical patient management¹⁷.

Over the past ten years, various other methods for MRD assessment in solid tumors including NSCLC have been explored¹⁸⁻²⁰ but their clinical significance has remained unclear because unlike MAGE-A based MRD assessment in resected NSCLC, none of these has been investigated in a prospective clinical study on the predictive value for the survival of patients. The potential of MAGE-A based MRD assessment for clinical decision making in resected (or otherwise removed) solid tumors, however, does not only lie in the strong statistical correlation with survival but also in the biological role of MAGE-A proteins as promoters of malignancy that inhibit p53-mediated apoptosis and senescence and confer resistance to chemotherapeutic drugs that act via p53-mediated apoptosis¹². Accordingly, MAGE-A based MRD assessment may serve two purposes: Better patient selection for adjuvant therapy, which is urgently needed especially in the clinical management of elderly patients²¹, and better selection of the kind of adjuvant therapy, which has the highest probability of best efficacy against MRD.

On the one hand, patients with completely resected (or otherwise removed) NSCLC (or other solid tumors) who present with MAGE-A expression levels ≧0.2 in blood or bone marrow are likely to benefit from adjuvant systemic therapy irrespective of tumor size and lymph node status because of their independent high risk of developing distant metastasis (hazard ratio 4.03). On the other hand, unnecessary toxic treatment such as adjuvant systemic chemotherapy may be avoidable in (elderly) patients with MAGE-A expression levels below 0.2, who are unlikely to develop distant metastasis (5-year distant-metastasis-free survival 87%). In particular for tumor patients suffering from co-morbidities and/or for elderly tumor patients there is a high need for reliable prediction that development of distant metastasis is unlikely in order to exempt them from a subsequent therapy and its side effects thus keeping an optimal benefit to risk balance and maximizing quality of life.

As to the selection of the kind of adjuvant therapy, the potential benefit from adjuvant treatment of systemic MRD diagnosed through MAGE-A expression measurement by chemotherapeutic drugs that act via p53-mediated apoptosis is expected to be small because MAGE-A proteins inhibit p53-mediated apoptosis¹¹. On the contrary, MAGE-A based MRD assessment may be supportive of other adjuvant treatment modalities such as adjuvant vaccination with MAGE-A protein. MAGE-A proteins were originally discovered as target antigens of cytotoxic T cells in malignant melanoma²² and adjuvant vaccination with a recombinant MAGE-A3 fusion protein in resected MAGE-A3 positive NSCLC has been reported to significantly improve disease-free and overall survival²³. Because of the high level of sequence identity among the different MAGE-A proteins especially within the so-called MAGE Homology Domain (MHD) comprising approximately 170 amino acids 10, the MAGE-A3 protein vaccine may have induced cytototoxic T cells not only recognizing MAGE-A3 but also other MAGE-A antigens. Considering the critical role of MAGE-A positive MRD for the development of distant metastasis in resected NSCLC as demonstrated by the prospective clinical study reported here, the efficacy of the adjuvant MAGE-A3 vaccine was most likely attributed to the induced MAGE-A specific cytotoxic T cells having destroyed MAGE-A positive disseminated NSCLC cells, that otherwise would have grown out into distant metastases. In this respect, measuring MAGE-A expression levels in bone marrow and blood may not only help to predict survival of patients after tumor removal but may also serve as a biomarker to monitor reduction or clearance of systemic MRD under adjuvant therapy.

Statistical Analysis.

The statistical calculations were performed using SPSS software (version 21.0 for PC, IBM Inc.). To analyze a possible association of bone marrow and blood findings with clinicopathological variables, the two-tailed Pearson's χ² test or Fisher's exact test in frequencies <5 were used. The threshold for statistical significance was p<0.05. The primary outcomes—overall survival, cancer-free survival and distant-disease-free interval—were characterized using Kaplan-Meier plots and survival distributions were compared by log-rank statistics.

The joint effects of other prognostically relevant variables were further examined using the Cox proportional hazard model. The respective covariables were entered stepwise forward into the model to assess possible independence of the prognostic value of MAGE-A transcripts. The 0.05 level of significance was used for entering or removing a covariable from this model.

TABLE 2
MAGE-A expression in bone marrow or blood according
to clinical and pathological characteristics

| | | Patients with MAGE-A | | Patients with MAGE-A | |
|---|---|---|---|---|---|
| | All | positive bone marrow | | positive bone marrow | |
| | study | or blood at or above | | or blood at expression | |
| | patients | LLOQ | | level ≧0.2 | |
| Characteristic | N = 94 | N = 41 (43.6%) | p value^{a} | N = 29 (30.9%) | p value^{a} |
| Tumor extension | | | | | |
| pT1-pT2 | 64 | 29 (47.5%) | | 20 (31.2%) | |
| pT3-pT4 | 30 | 12 (40.0%) | 0.63 | 9 (30.0%) | 0.90 |
| Lymph node status | | | | | |
| pN0-1 | 72 | 34 (47.2%) | | 23 (31.9%) | |
| pN2 | 22 | 7 (31.8%) | 0.20 | 6 (27.3%) | 0.68 |
| Tumor histology | | | | | |
| Adeno | 37 | 19 (51.4%) | | 14 (37.9%) | |
| Squamous | 42 | 18 (42.9%) | | 13 (31.0%) | |
| Miscellaneous^{b} | 15 | 4 (26.7%) | 0.26 | 2 (13.3%) | 0.22 |
| Grading | | | | | |
| G1-G2 | 40 | 18 (45.0%) | | 14 (35.0%) | |
| G3-G4 | 54 | 23 (42.6%) | 0.82 | 15 (27.8%) | 0.45 |
| Age | | | | | |
| ≦66 years | 50 | 18 (36.0%) | | 16 (32.0%) | |
| >66 years | 44 | 23 (52.3%) | 0.11 | 13 (29.5%) | 0.90 |

^{a}Two-sided p values determined by Pearson's Chi square test show possible significance of correlation between detection of MAGE-A transcripts and clinicopathological parameters. LLOQ = lower limit of quantification.
^{b}“Miscellaneous^{”} represents 8 adenosquamous carcinomas and 7 large cell carcinomas.

TABLE 3
Treatment failure according to MAGE-A expression level
in bone marrow or blood and site of recurrence

| | | Patients with MAGE-A | Patients with MAGE-A |
|---|---|---|---|
| | | positive bone marrow | positive bone marrow |
| | Total | or blood at expression | or blood at expression |
| | Cohort | level ≧0.2 | level <0.2 |
| Variable | N = 89 | N = 29 (32.6%) | N = 60 (67.4%) |
| Disease | 40 | 20 | 20 |
| recurrence | | | |
| Local | 16 | 6 | 10 |
| recurrence | | | |
| Distant metastasis | 30 | 16 | 14 |
| Distant metastasis | 24 | 14 | 10 |
| without prior local | | | |
| recurrence | | | |
| Death | 49 | 22 | 27 |
| Death of | 49 | 22 | 27 |
| any cause | | | |
| Cancer-related | 36 | 18 | 18 |
| death | | | |
| Event-free | 40 | 7 | 33 |
| outcome | | | |

TABLE 4
MAGE-A expression levels by quantitative multimarker MAGE-A RT-PCR
in blood and bone marrow samples of selected NSCLC patients

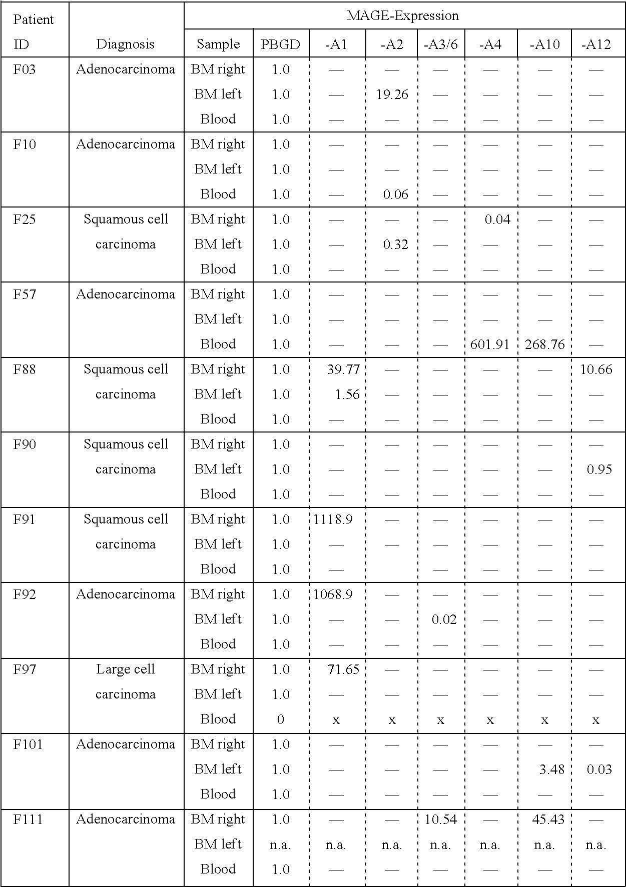

BM: bone marrow;
n.a.: not available;
shadowed: positive signal at expression level ≧ 0.2;
—: signal below detection limit;
x: not evaluable due to absence of PBGD signal (i.e., housekeeping gene);
PBGD transcript amplified: 1.0;
no PBGD transcript amplified: 0.

TABLE 5
Univariate analysis of distant metastasis-free survival with different
threshold levels for MAGE-A expression in bone marrow or blood

| MAGE-A expression | |
|---|---|
| threshold | p-value |
| ≧0.01 vs. <0.01 | p = 0.049 |
| ≧0.05 vs. <0.05 | p = 0.013 |
| ≧0.1 vs. <0.1 | p = 0.002 |
| ≧0.2 vs. <0.2 | p < 0.001 |
| ≧0.3 vs <0.3 | p = 0.004 |
| ≧0.5 vs. <0.5 | p = 0.013 |
| ≧1.0 vs. <1.0 | p = 0.09 |

* p-values of univariate analyses were determined by log-rank test.

TABLE 6
Multivariable hazard ratios for overall survival, cancer-free survival and distant metastasis-free survival

| | Overall survival | | | Cancer-free survival | | | Distant metastasis-free survival | | |
|---|---|---|---|---|---|---|---|---|---|
| | univariate | multivariate | Hazard | univariate | multivariate | Hazard | univariate | multivariate | Hazard |
| | analysis | analysis | Ratio | analysis | analysis | Ratio | analysis | analysis | Ratio |
| Variable | (p Value)^{a} | (p Value)^{b} | (95% CI) | (p Value)^{a} | (p Value)^{b} | (95% CI) | (p Value)^{a} | (p Value)^{b} | (95% CI) |
| MAGE-A gene expression | 0.007 | 0.002 | 2.56 | 0.002 | 0.001 | 3.32 | <0.001 | 0.001 | 4.03 |
| (≧0.2 vs. <0.2) | | | (1.42-4.63) | | | (1.66-6.61) | | | (1.77-9.18) |
| Tumor size | <0.001 | 0.002 | 1.71 | 0.007 | 0.016 | 1.65 | 0.51 | n.s. | — ^{c} |
| (T₃₋₄ vs. T₁₋₂) | | | (1.23-2.38) | | | (1.11-2.45) | | | |
| Lymph node status | 0.005 | 0.057 | 1.39 | 0.003 | 0.026 | 1.56 | 0.30 | n.s. | — ^{c} |
| (N₂ vs. N₀₋₁) | | | (1.00-1.93) | | | (1.07-2.29) | | | |
| Grading | 0.17 | n.s. | — ^{c} | 0.30 | n.s. | — ^{c} | 0.77 | n.s. | — ^{c} |
| (G3-4 vs. G1-2) | | | | | | | | | |
| patient age | 0.94 | n.s. | — ^{c} | 0.67 | n.s. | — ^{c} | 0.84 | n.s. | — ^{c} |
| (>66 vs. ≦66 years) | | | | | | | | | |
| Tumor histology | 0.60 | n.s. | — ^{c} | 0.78 | n.s. | — ^{c} | 0.45 | n.s. | — ^{c} |
| (squamous carcinoma vs. | | | | | | | | | |
| adenocarcinoma vs. | | | | | | | | | |
| Miscellaneous) | | | | | | | | | |

^{a}P-values of univariate analyses were determined by log-rank test.
^{b}Stepwise multivariate analysis was performed using the Cox proportional-hazard model.
^{c} No estimate of relative risk is given, since the variable was not significant on multivariate analysis.
n.s. = not significant

### D. REFERENCE LISTING

The following references, to the extent that they provide exemplary procedural or other details supplementary to those set forth herein, are specifically incorporated herein by reference.
1. Siegel R, Ward E, Brawley O, Jemal A. Cancer statistics, 2011: the impact of eliminating socioeconomic and racial disparities on premature cancer deaths. CA Cancer J Clin 2011; 61(4): 212-36.
2. Wahbah M, Boroumand N, Castro C, El-Zeky F, Eltorky M. Changing trends in the distribution of the histologic types of lung cancer: a review of 4,439 cases. Ann Diagn Pathol 2007; 11(2): 89-96.
3. Goldstraw P, Crowley J, Chansky K, et al. The IASLC Lung Cancer Staging Project: proposals for the revision of the TNM stage groupings in the forthcoming (seventh) edition of the TNM Classification of malignant tumours. J Thorac Oncol 2007; 2(8): 706-14.
4. Bruggemann M, Raff T, Flohr T, et al. Clinical significance of minimal residual disease quantification in adult patients with standard-risk acute lymphoblastic leukemia. Blood 2006; 107(3): 1116-23.
5. Kufer P, Zippelius A, Lutterbuse R, et al. Heterogeneous expression of MAGE-A genes in occult disseminated tumor cells: a novel multimarker reverse transcription-polymerase chain reaction for diagnosis of micrometastatic disease. Cancer Res 2002; 62(1): 251-61.
6. De Plaen E, Arden K, Traversari C, et al. Structure, chromosomal localization, and expression of 12 genes of the MAGE family. Immunogenetics 1994; 40(5): 360-9.
7. Rogner U C, Wilke K, Steck E, Korn B, Poustka A. The melanoma antigen gene (MAGE) family is clustered in the chromosomal band Xq28. Genomics 1995; 29(3): 725-31.
8. Chomez P, De Backer O, Bertrand M, De Plaen E, Boon T, Lucas S. An overview of the MAGE gene family with the identification of all human members of the family. Cancer Res 2001; 61(14): 5544-51.
9. Gjerstorff M F, Ditzel H J. An overview of the GAGE cancer/testis antigen family with the inclusion of newly identified members. Tissue Antigens 2008; 71(3): 187-92.
10. Meek D W, Marcar L. MAGE-A antigens as targets in tumour therapy. Cancer Lett 2012; 324(2): 126-32.
11. Ladelfa M F, Peche L Y, Toledo M F, Laiseca J E, Schneider C, Monte M. Tumor-specific MAGE proteins as regulators of p53 function. Cancer Lett 2012; 325(1): 11-7.
12. Monte M, Simonatto M, Peche L Y, et al. MAGE-A tumor antigens target p53 transactivation function through histone deacetylase recruitment and confer resistance to chemotherapeutic agents. Proc Natl Acad Sci USA 2006; 103(30): 11160-5.
13. Kwon S, Kang S H, Ro J, Jeon C H, Park J W, Lee E S. The melanoma antigen gene as a surveillance marker for the detection of circulating tumor cells in patients with breast carcinoma. Cancer 2005; 104(2): 251-6.
14. Mecklenburg I, Weckermann D, Zippelius A, et al. A multimarker real-time RT-PCR for MAGE-A gene expression allows sensitive detection and quantification of the minimal systemic tumor load in patients with localized cancer. J Immunol Methods 2007; 323(2): 180-93.
15. Jacques J, Hill D P, Shier K J, Jindani A, Miller A B. Appraisal of the World Health Organization classification of lung tumours. Can Med Assoc J 1980; 122(8): 897-901.
16. Sobin L H WC. International Union Against Cancer (UICC), TNM Classification of Malignant Tumours, 6th ed. New-York: Wiley-Liss; 2002.
17. Hokland P, Ommen H B. Towards individualized follow-up in adult acute myeloid leukemia in remission. Blood 2011; 117(9): 2577-84.
18. Pantel K, Izbicki J, Passlick B, et al. Frequency and prognostic significance of isolated tumour cells in bone marrow of patients with non-small-cell lung cancer without overt metastases. Lancet 1996; 347(9002): 649-53.
19. Cote R J, Beattie E J, Chaiwun B, et al. Detection of occult bone marrow micrometastases in patients with operable lung carcinoma. Ann Surg 1995; 222(4): 415-23; discussion 23-5.
20. Kubuschok B, Passlick B, Izbicki J R, Thetter O, Pantel K. Disseminated tumor cells in lymph nodes as a determinant for survival in surgically resected non-small-cell lung cancer. J Clin Oncol 1999; 17(1): 19-24.
21. Hurria A, Togawa K, Mohile S G, et al. Predicting chemotherapy toxicity in older adults with cancer: a prospective multicenter study. J Clin Oncol 2011; 29(25): 3457-65.
22. van der Bruggen P, Traversari C, Chomez P, et al. A gene encoding an antigen recognized by cytolytic T lymphocytes on a human melanoma. Science 1991; 254(5038): 1643-7.
23. Decoster L, Wauters I, Vansteenkiste J F. Vaccination therapy for non-small-cell lung cancer: review of agents in phase III development. Ann Oncol 2012; 23(6): 1387-93.
24. Sang M, Wang L, Ding C, et al. Melanoma-associated antigen genes—an update. Cancer Lett 2011; 302(2): 85-90.
25. Sienel W, Mecklenburg I, Dango S, ET AL. Detection of MAGE-A Transcripts in Bone Marrow Is an Independent Prognostic Factor in Operable Non-Small-Cell Lung Cancer. Clin Cancer Res 2007; 13(13): 3840-7.
26. Vantomme V, Boel P, De Plaen E, Boon T, van der Bruggen P: A new tumor-specific antigenic peptide encoded by MAGE-6 is presented to cytolytic T lymphocytes by HLA-Cw16. Cancer Immun 2003 Dec. 10; 3:17.
27. Martelange V, De Smet C, De Plaen E, Lurquin C, Boon T: Identification on a human sarcoma of two new genes with tumor-specific expression. Cancer Res 2000 Jul. 15; 60(14):3848-55.

1. A method of treating a patient or exempting a patient from further treatment subsequent to tumor removal such as surgery comprising the following steps:
a) performing a highly sensitive real-time PCR for specific detection of transcripts (mRNA) of more than one MAGE gene and a real-time PCR for specific detection of transcripts (mRNA) of a reference gene such as porphobilinogen desaminase (PBGD), glyceraldehyd-3-phosphate dehydrogenase (GAPDH), beta-2-microglobin or beta-actin in a tissue sample of a tumor patient, wherein reverse transcription of mRNA into cDNA of more than one MAGE gene and of the reference gene prior to real-time PCR is carried out in the same cDNA synthesis reaction
b) performing said highly sensitive real-time PCR for more than one MAGE gene and said real-time PCR for a reference gene such as porphobilinogen desaminase (PBGD), glyceraldehyd-3-phosphate dehydrogenase (GAPDH), beta-2-microglobin or beta-actin in at least one calibrator sample
c) quantifying the expression level of said MAGE genes in said tissue sample of a tumor patient by calculating the calibrator normalized relative ratio according to the following formula:
$$Normalized Ratio = \frac{\frac{{N}_{To ( S )}}{{N}_{Ro ( S )}}}{\frac{{N}_{To ( C )}}{{N}_{Ro ( C )}}} = \frac{\frac{{E}_{T}^{CpT ( C )}}{{E}_{R}^{CpR ( C )}}}{\frac{{E}_{T}^{CpT ( S )}}{{E}_{R}^{CpR ( S )}}} = \frac{{E}_{T}^{CpT ( C )}}{{E}_{R}^{CpR ( C )}} \times \frac{{E}_{R}^{CpR ( S )}}{{E}_{T}^{CpT ( S )}} = {E}_{T}^{CpT ( C ) - CpT ( S )} \times {E}_{R}^{CpR ( S ) - CpR ( C )}$$
N_{To}/N_{Ro}: Initial number of target/reference molecules, i.e., of MAGE cDNAs/reference cDNA
CpT: Cycle number at target detection threshold (crossing point)
CpR: Cycle number at reference detection threshold (crossing point)
E_{T}: Efficiency of target amplification
E_{R}: Efficiency of reference amplification
T: Target (i.e., MAGE gene (s))
R: Reference (i.e., reference gene)
S: Tissue sample of a tumor patient
C: Calibrator sample
d) performing a subsequent therapy, if the resulting MAGE gene expression level of step c) is equal to or above a threshold value selected from the interval of 0.01 to 1.0, preferably selected from the interval of 0.05 to 0.5, more preferably selected from the interval of 0.1 to 0.3 and most preferably is equal to or above a threshold value of 0.2, and/or
e) exempting patients from a subsequent therapy, if the resulting MAGE gene expression level of step c) is below a threshold value selected from the interval of 0.01 to 1.0, preferably selected from the interval of 0.05 to 0.5, more preferably selected from the interval of 0.1 to 0.3 and most preferably is below 0.2.

2. The method of claim 1, wherein the MAGE genes are selected from the functional genes of MAGE subfamilies A, B and/or C, in particular wherein the MAGE genes comprise MAGE-A 1, 2, 3, 4, 6, 10 and/or 12.

3. The method of claim 1, wherein at least one primer for reverse transcription of MAGE mRNA is selected from at least group A of the following groups of oligonucleotides:

| | | SEQ |
|---|---|---|
| primer | sequence (5′-3′) | ID NO: |
| (A) | | |
| MgRT1a | CCA GCA TTT CTG CCT TTG TGA | 1 |
| MgRT1b | CCA GCA TTT CTG CCT GTT TG | 2 |
| MgRT2 | CAG CTC CTC CCA GAT TT | 3 |
| MgRT3a | ACC TGC CGG TAC TCC AGG | 4 |
| MgRT3b | ACC TGC CGG TAC TCC AGG TA | 5 |
| MgRT4 | GCC CTT GGA CCC CAC AGG AA | 6 |
| MgRT5a | AGG ACT TTC ACA TAG CTG GTT TCA | 7 |
| MgRT5b | GGA CTT TCA CAT AGC TGG TTT C | 8 |
| MgRT6 | TTT ATT CAG ATT TAA TTT C | 9 |
| (B) | | |
| Mg1_RT1 | CAA GAG ACA TGA TGA CTC TC | 10 |
| Mg1_RT2 | TTC CTC AGG CTT GCA GTG CA | 11 |
| Mg1_RT3 | GAG AGG AGG AGG AGG TGG C | 12 |
| Mg1_RT4 | GAT CTG TTG ACC CAG CAG TG | 13 |
| Mg1_RT5a | CAC TGG GTT GCC TCT GTC | 14 |
| Mg1_RT5c | CTG GGT TGC CTC TGT CGA G | 15 |
| Mg1_RT5d | GGG TTG CCT CTG TCG AGT G | 16 |
| Mg1_RT5e | GGC TGC TGG AAC CCT CAC | 17 |
| Mg1_RT6 | GCT TGG CCC CTC CTC TTC AC | 18 |
| Mg1_RT7 | GAA CAA GGA CTC CAG GAT AC | 19 |

4. The method of claim 1, wherein the primer for reverse transcription of PBGD mRNA is selected from the following group of oligonucleotides:

| | | SEQ |
|---|---|---|
| primer | Sequence (5′-3′) | ID NO: |
| PBGD_RT2 | CAT ACA TGC ATT CCT CAG GGT | 20 |
| PBGD_RT3 | GAA CTT TCT CTG CAG CTG GGC | 21 |
| PBGD_RT4 | TGG CAG GGT TTC TAG GGT CT | 22 |
| PBGD_RT10a | GGT TTC CCC GAA TAC TCC TG | 23 |
| PBGD_RT10d | TTG CTA GGA TGA TGG CAC TG | 24 |
| PBGD_RT12b | CCA AGA TGT CCT GGT CCT TG | 25 |
| PBGD_RT12c | CAG CAC ACC CAC CAG ATC | 26 |
| PBGD_RT12d | AGA GTC TCG GGA TCG TGC | 27 |
| PBGD_RT12e | AGT CTC GGG ATC GTG CAG | 28 |
| PBGD_RT12f | TCT CGG GAT CGT GCA GCA | 29 |
| PBGD_RT12g | ATG CAG CGA AGC AGA GTC T | 30 |
| PBGD_RT12h | CCT TTC AGC GAT GCA GCG | 31 |
| PBGD_RT13a | GTA TGC ACG GCT ACT GGC | 32 |
| PBGD_RT14a | GCT ATC TGA GCC GTC TAG AC | 33 |
| PBGD_RT15a | AAT GTT ACG AGC AGT GAT GC | 34 |
| PBGD_RT15b | TGG GGC CCT CGT GGA ATG | 35 |
| PBGD_RT15e | CAG TTA ATG GGC ATC GTT AAG | 36 |
| PBGD_RT15f | ATC TGT GCC CCA CAA ACC AG | 37 |
| PBGD_RT15g | GGC CCG GGA TGT AGG CAC | 38 |
| PBGD_RT15h | GGT AAT CAC TCC CCA GAT AG | 39 |
| PBGD_RT15i | CTC CCG GGG TAA TCA CTC | 40 |
| PBGD_RT15j | CAG TCT CCC GGG GTA ATC | 41 |
| PBGD_RT15k | TGA GGA GGC AAG GCA GTC | 42 |
| PBGD_RT15l | GGA TTG GTT ACA TTC AAA GGC | 43 |

5. The method of claim 1, wherein the PCR-primers for amplification of PBGD-cDNA comprise oligonucleotides selected from the following groups:

| PBGD sense primer | sequence (5′-3′) | SEQ ID NO: |
|---|---|---|
| hu_PBGD_se | AGA GTG ATT CGC GTG GGT ACC | 44 |
| PBGD_8 | GGC TGC AAC GGC GGA AGA AAA C | 45 |
| PBGD_8_F | TGC AAC GGC GGA AGA AAA C | 46 |
| PBGD_ATG-Eco | ATG TCT GGT AAC GGC AAT GC | 47 |
| PBGD antisense | | |
| primer | sequence (5′-3′) | SEQ ID NO: |
| PBGD_3 | TTG CAG ATG GCT CCG ATG GTG AA | 48 |
| PBGD_3.1_R | GGC TCC GAT GGT GAA GCC | 49 |
| PBGD_R | TTG GGT GAA AGA CAA CAG CAT C | 50 |

in particular wherein the primer pairs hu_PBGD_se and PBGD_3.1_R or hu_PBGD_se and PBGD_R are used for PCR-amplification of PBGD-cDNA.

6. The method of claim 1, wherein in total not more than two different oligonucleotides, in particular MgRT_3a and PBGD_RT15b, are used as primers for reverse transcription in the cDNA-synthesis reaction.

7. The method of claim 1, wherein the MAGE- and/or the calibrator-PCR are nested or semi-nested PCRs.

8. The method of claim 1, wherein PCR-primers are used (i) comprising pairs of oligonucleotides specifically amplifying only a single member of the selected group of MAGE genes, respectively, or (ii) comprising pairs of oligonucleotides amplifying more than one member of the selected group of MAGE genes, respectively.

9. The method of claim 8, wherein the method is carried out with a single or double pair of PCR-primers amplifying all members of the selected group of MAGE genes, respectively.

10. The method of claim 1, wherein the PCR-primers for amplification of MAGE-cDNA comprise oligonucleotides selected from one of the following groups:

| | | SEQ |
|---|---|---|
| PCR-primer | sequence (5′-3′) | ID NO: |
| (C) | | |
| MAGE-A1 | GTA GAG TTC GGC CGA AGG AAC | 51 |
| MAGE-A1 | CAG GAG CTG GGC AAT GAA GAC | 52 |
| MAGE-A2 | CAT TGA AGG AGA AGA TCT GCC T | 53 |
| MAGE-A2 | GAG TAG AAG AGG AAG AAG CGG T | 54 |
| MAGE-A3/6 | GAA GCC GGC CCA GGC TCG | 55 |
| MAGE-A3/6 | GAT GAC TCT GGT CAG GGC AA | 56 |
| MAGE-A4 | CAC CAA GGA GAA GAT CTG CCT | 57 |
| MAGE-A4 | TCC TCA GTA GTA GGA GCC TGT | 58 |
| MAGE-A10 | CTA CAG ACA CAG TGG GTC GC | 59 |
| MAGE-A10 | GCT TGG TAT TAG AGG ATA GCA G | 60 |
| MAGE-A12 | TCC GTG AGG AGG CAA GGT TC | 61 |
| MAGE-A12 | ATC GGA TTG ACT CCA GAG AGT A | 62 |
| (D) | | |
| MAGE-A1 | TAG AGT TCG GCC GAA GGA AC | 63 |
| MAGE-A1 | CTG GGC AAT GAA GAC CCA CA | 64 |
| MAGE-A2 | CAT TGA AGG AGA AGA TCT GCC T | 65 |
| MAGE-A2 | CAG GCT TGC AGT GCT GAC TC | 66 |
| MAGE-A3/6 | GGC TCG GTG AGG AGG CAA G | 67 |
| MAGE-A3/6 | GAT GAC TCT GGT CAG GGC AA | 68 |
| MAGE-A4 | CAC CAA GGA GAA GAT CTG CCT | 69 |
| MAGE-A4 | CAG GCT TGC AGT GCT GAC TCT | 70 |
| MAGE-A10 | ATC TGA CAA GAG TCC AGG TTC | 71 |
| MAGE-A10 | CGC TGA CGC TTT GGA GCT C | 72 |
| MAGE-A12 | TCC GTG AGG AGG CAA GGT TC | 73 |
| MAGE-A12 | GAG CCT GCG CAC CCA CCA A | 74 |

in particular a primer of group C for the first round and/or a primer of group D for a second round of PCR-amplification.

11. The method of claim 1, wherein the at least one calibrator sample is any tissue of a healthy subject spiked with a defined number of tumor cells and/or is subjected to reverse transcription of mRNA into cDNA of more than one MAGE gene and of the reference gene in the same cDNA synthesis reaction followed by said highly sensitive real-time PCR for more than one MAGE gene and said real-time PCR for a reference gene.

12. The method of claim 1, wherein said tissue sample of said tumor patient and/or the at least one calibrator sample of a healthy subject is bone marrow or blood.

13. The method of claim 1, wherein the at least one calibrator sample of a healthy subject is spiked with a defined number of tumor cells of the human melanoma cell line Mz2-Mel or the human sarcoma cell line LB23-SAR.
